SEQUENCE LISTING (2 sequences)

SEQ ID NO: 1 - protein, 133 aa, Arabidopsis thaliana
MGGALSTVFGSGEDATAAGTESEPSRVLKFSSSARWQLHFNEIKESNKLLVVDFSASWCGPCRMIEPAIHAMADKFNDVDFVKLDVDELPDVAKEFNVTAMPTFVLVKRGKEIERIIGAKKDELEKKVSKLRA

SEQ ID NO: 2 - protein, 213 aa, Arabidopsis thaliana
MNSFSAFSEMFGSDYEPQGGDYCPTLATSCPKKPAGRKKFRETRHPIYRGVRQRNSGKWVSEVREPNKKTRIWLGTFQTAEMAARAHDVAALALRGRSACLNFADSAWRLRIPESTCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

(19) United States
(12) Patent Application Publication
(10) Pub. No.: US 2023/0374534 A1
(43) Pub. Date: Nov. 23, 2023

(54) TRANSGENIC PLANT HAVING COLD RESISTANCE OVEREXPRESSING THIOREDOXIN TRX-H2 PROTEIN

(71) Applicant: INDUSTRY-ACADEMIC COOPERATION FOUNDATION GYEONGSANG NATIONAL UNIVERSITY, Gyeongsangnam-do (KR)

(72) Inventors: Sang Yeol LEE, Gyeongsangnam-do (KR); Eun Seon LEE, Gyeongsangnam-do (KR); Joung Hun PARK, Gyeongsangnam-do (KR); Seong Dong WI, Gyeongsangnam-do (KR); Chang Ho KANG, Gyeongsangnam-do (KR); Ho Byoung CHAE, Gyeongsangnam-do (KR); Seol Ki PAENG, Gyeongsangnam-do (KR); Su Bin BAE, Gyeongsangnam-do (KR)

(21) Appl. No.: 18/028,313

(22) PCT Filed: Sep. 9, 2021
(86) PCT No.: PCT/KR2021/012252

§ 371 Date: Mar. 24, 2023

(30) Foreign Application Priority Data
Sep. 25, 2020 (KR) 10-2020-0124428

(51) Int. Cl. C12N 15/82; C07K 14/415

(52) U.S. Cl. CPC C12N 15/8273; C07K 14/415

(57) ABSTRACT

A transgenic plant having cold resistance overexpressing thioredoxin Trx-h2 protein. More specifically, the present invention relates to a transgenic plant having cold resistance overexpressing thioredoxin Trx-h2 protein which is an interacting partner of cold-responsive C-repeat-binding transcription factors (CBF 1).

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/012252, filed Sep. 9, 2021, which claims priority to the benefit of Korean Patent Application No. 10-2020-0124428 filed in the Korean Intellectual Property Office on Sep. 25, 2020, the entire contents of which are incorporated herein by reference.

### BACKGROUND

### 1. Technical Field

The present invention relates to a transgenic plant having cold resistance overexpressing thioredoxin Trx-h2 protein. More specifically, the present invention relates to a transgenic plant having cold resistance(tolerance) overexpressing thioredoxin Trx-h2 protein which is an interacting partner of cold-responsive C-repeat-binding transcription factors (CBF 1).

### 2. Background Art

Cold stress restricts plant growth and development and limits the productivity of agricultural crops. To cope with low temperatures and to acquire freezing tolerance, plants have developed a mechanism known as cold acclimation, whereby plants undergo massive reprogramming of the cellular metabolism and remodeling of the tissue architecture when exposed to low non-freezing temperatures. Genes encoding C-repeat binding factors (CBFs), transcription factors belonging to the APETALA2/ETHYLENE RESPONSE FACTOR (AP2/ERF) family, play the most important role in cold acclimation. However, the expression of CBF genes is regulated not only by cold-signaling factors but also by various phytohormones, suggesting that CBFs serve as a hub for the crosstalk between cold- and hormone-signaling pathways and perform multiple roles in the regulation of plant growth and cold tolerance.

Ectopic overexpression of CBFs strongly enhances the freezing tolerance of plants but is detrimental to plant growth. Moreover, cbf1-3 triple mutant Arabidopsis (hereafter referred to as cbfs) exhibit high sensitivity to freezing, poor seed germination, and stress-sensitivity under ambient temperatures. These data suggest that the expression of CBFs must stay under a critical threshold at normal condition and the CBF activity should be precisely controlled for plant growth and cold tolerance at different temperatures. Despite extensive studies on the transcriptional control of CBF expression under cold conditions, numerous aspects of CBF activity regulation remain elusive.

To understand how CBF activity is regulated, we examined the expression level and protein structure of CBFs in Arabidopsis thaliana (Col-0 ecotype; wild type) with or without cold treatment (FIG. 1A). Total proteins extracted from Col-0 were separated by SDS-PAGE on reducing/non-reducing gels and subjected to western blotting using anti-CBFs antibody prepared in our laboratory. While the level of CBFs was noticeable at warm temperature (22° C.), the amount of protein increased with the duration of cold exposure (4° C.), as estimated by a reducing gel (FIG. 1A; lower panel). However, the non-reducing gel mainly showed the high molecular weight (HMW) band of CBFs (representing oligomers) at 22° C. (FIG. 1A; upper panel, lane 1), indicating that CBFs exist predominantly as HMW oligomers. By contrast, the amounts of middle-sized CBF oligomers and monomer increased significantly at 4° C., while that of HMW oligomer decreased (lanes 2-4). The result suggests that CBF monomers are produced both from cold-dependent dissociation of high-oligomers and cold-induced newly synthesized CBFs. This cold-dependent structural switching from HMW oligomers to middle-sized oligomer and monomers appears to represent a very early response of plant to a cold snap and their ability to induce cold acclimation.

We hypothesized that the structural switching of CBFs may be controlled by their interacting partners. Therefore, to identify interacting partners of CBFs, we performed immunoprecipitation (IP), followed by mass spectrometry analysis, using total proteins extracted from Arabidopsis CBF1-Myc^{OE} plants (FIGS. 5A and 5B) exposed to 4° C. for 6 h.

Thus, the present inventors revealed that unlike the Arabidopsis trx-h2 knock-out mutant, Trx-h2 overexpression lines were highly cold tolerant. Our findings reveal the mechanism by which cold-mediated redox changes induce low temperature-dependent structural switching from high molecular weight oligomers to the middle-sized oligomers and monomer and functional activation of CBFs, thus conferring plant cold tolerance. Accordingly, the present invention is completed.

### SUMMARY

The technical problems to be solved is to confirm that unlike the Arabidopsis trx-h2 knock-out mutant, Trx-h2 overexpression lines were highly cold tolerant. Our findings reveal the mechanism by which cold-mediated redox changes induce low temperature-dependent structural switching from high molecular weight oligomers to the middle-sized oligomers and monomer and functional activation of CBFs, thus conferring plant cold tolerance(resistance).

The present invention provides a transgenic plant having cold resistance overexpressing thioredoxin Trx-h2 protein having an amino acid sequence of SEQ ID NO: 1, wherein the thioredoxin Trx-h2 protein interacts with C-repeat-binding transcription factor 1 (CBF 1) having an amino acid sequence of SEQ ID NO: 2 to switch CBF 1 from high molecular weight oligomers to low molecular weight monomers at low temperatures of 0 to 10° C., thus conferring plant cold resistance.

Also, the thioredoxin Trx-h2 protein is myristoylated in the cytoplasm by glycine, the second amino acid residue, but it is demyristoylated from the cytoplasm at low temperatures of 0 to 10° C., and then it is translocated to a nucleus, interacting with CBF1 to cause structural switching of CBF1, thus conferring plant cold resistance.

Also, the thioredoxin Trx-h2 protein has a well-conserved Trx motif with 122 amino acids from the 31st residue to the 133rd residue, and cysteine, the 59th and 62nd residues in the Trx motif, interacts with CBF 1.

Also, the Cysteine, the 23, 30, 100, 117, and 137th conserved residues in the CBF1, interacts with the thioredoxin Trx-h2 protein.

The present invention provides a Trx-h2 overexpression plant with highly cold tolerance, unlike the Arabidopsis trx-h2 knockout mutant. The present invention provides a Trx-h2 overexpression plant with highly cold tolerant in which cold-mediated redox changes induce low temperature-dependent structural switching from high molecular weight oligomers to the middle-sized oligomers and monomer and functional activation of CBFs.

### BRIEF DESCRIPTION OF THE DRAWINGS

FIGS. 1A to 1I show that cold-mediated demyristoylation of cytoplasmic Trx-h2 enables its translocation to the nucleus and subsequent interaction with and dissociation of CBF oligomers.

FIG. 1A shows the cold-induced CBFs expression and structural switching from oligomeric to monomeric forms in Col-0 plant at 4° C. Total proteins were extracted from plants incubated at 4° C. for the indicated time points, and CBFs level and structural changes were analyzed by SDS-PAGE on reducing (lower panel) and non-reducing (upper panel) gels by western blotting. Labels ‘O’ and ‘M’ indicate CBF oligomers and monomer, respectively. FIG. 1B, In vitro pull-down assay of Trx-h2 with His-CBFs or His-MBP (control) analyzed by SDS-PAGE and western blotting. FIG. 1C, Co-immunoprecipitation (Co-IP) assay of CBF-Myc extracted from CBF-Myc^{OE} plants incubated at 22° C. or 4° C. for 6 h. FIG. 1D, BiFC assay. Trx-h2-YN and YC-CBF were co-expressed in tobacco leaves and incubated at 4° C. or 22° C. for 6 h. FIG. 1E, Subcellular location of Trx-h2-YFP in tobacco leaves incubated at 22° C. or 4° C. for 6 h. FIG. 1F, Role of Gly2 in the translocation of Trx-h2 to the nucleus. Trx-h2-YFP or Trx-h2(G/A)-YFP was expressed in tobacco leaves and incubated at 22° C. In FIGS. 1D to 1F, YFP signal detected under a confocal microscope was merged with that of the nuclear marker, NLS-RFP (Merge). Bright-field images (Bright) and scale bars (20 μm) are indicated. FIG. 1G, Separation of nuclear and non-nuclear fractions of proteins extracted from Col-0 plants incubated at 22° C. or 4° C. Trx-h2 was detected by western blotting. Anti-histone-H3 and anti-PEPC antibodies were used as nuclear and non-nuclear protein markers. FIGS. 1H and 1I, Myristoylation of Trx-h2 in Trx-h2-V5^{OE}/trx-h2 plants incubated at 22° C. and 4° C. for 6 h (FIG. 1H) or Trx-h2(G/A)-V5^{OE/trx-h}2 plants at 22° C. (FIG. 1I) using the protocol (FIGS. 9A and 9B). The myristoylation of Trx-h2 did not cause a detectible mobility shift on SDS-PAGE gel.

FIGS. 2A to 2L show trx-h2-mediated reduction and monomerization of CBF1 in vitro and in vivo under cold stress.

FIGS. 2A and 2B show the western blot analysis of purified His-CBF1 incubated with 1 mM DTT or H₂O₂ (FIG. 2A) and with a Trx system containing 0.5 mM NADPH, 1 μM Trx reductase, and 5 μM Trx-h2 or Trx-h2(C/S) (FIG. 2B) at 25° C. for 30 min. Structural changes in CBF1 were analyzed by SDS-PAGE on non-reducing gels. FIGS. 2C and 2D show the western blot analysis of Trx-h2-mediated structural dissociation of CBF1 in CBF1-Myc^{OE}/Col-0 (FIG. 2C) and CBF1-Myc^{OE}/trx-h2 (FIG. 2D) plants exposed to cold. FIGS. 2E and 2F show the cold-induced dissociation of CBF1 oligomers into oxidized CBF1 monomers, which were converted into reduced monomers in P_{CBF1}:CBF1-Myc/Col-0 plants (FIG. 2E) but not in P_{CBF1}:CBF1-Myc/trx-h2 plants (FIG. 2F). Total proteins were treated with MM(PEG)24-methyl-PEG-Maleimide, and the reduced and oxidized forms of CBF1 were separated on non-reducing gels, followed by western blotting. Red(M) and Ox(M) represent reduced-( [symbol] ) and oxidized-( [symbol] ) monomeric bands, respectively. FIGS. 2G and 2H show that CBF1(C/S) is insensitive to redox changes in vitro (FIG. 2G) and in CBF1(C/S)-Myc-expressing tobacco leaves incubated at 4° C. (FIG. 2H). FIGS. 2I and 2J show the cold-induced cellular redox changes in Arabidopsis estimated by total glutathione levels (GSH+GSSG) (FIG. 2I) and GSH/GSSG ratio (FIG. 2J). FIGS. 2K and 2L show the structural switching of CBF1 in protein extracts of CBF1-Myc^{OE}/Col-0 (FIG. 2K) and CBF1-Myc^{OE}/trx-h2 (FIG. 2L) plants incubated with different ratios of GSH/GSSG for 1 h in a cell-free system. In FIGS. 2A-2L, ‘O’ and ‘M’ indicate CBF 1 oligomers and monomers.

FIGS. 3A to 3H show Trx-h2-dependent binding of CBF1 to COR15a promoter, and expression of COR genes under cold stress.

FIGS. 3A to 3D are the results of EMSAs showing the redox-dependent binding of His-CBF1 to biotin-labeled COR15a promoter fragment (oligoprobe) in the presence of 1 mM DTT (FIG. 3A), 1 mM H₂O₂ (FIG. 3B), and a Trx system comprising 5 μM Trx-h2 (FIG. 3C) or Trx-h2(C/S) (FIG. 3D) with His-MBP as a control. FIG. 3E is the schematic representation of the COR15a promoter. P1 and P2 indicate the primer binding sites for qRT-PCR. FIGS. 3F and 3G show that Chromatin immunoprecipitation-quantitative PCR (ChIP-qPCR) analysis for detecting the CBF1 binding to P1 and P2 regions of the COR15a promoter in various Arabidopsis genotypes exposed to 22° C. (FIG. 3F) or 4° C. (FIG. 3G) for 6 h. FIG. 3H shows qRT-PCR analysis of representative COR genes in Col-0, trx-h2, and trx-h3 (control) plants incubated at 4° C.

FIGS. 4A to 4J show that Trx-h2 enhances the freezing tolerance of Arabidopsis mainly through CBF signaling.

FIGS. 4A-4F show the comparison of the freezing tolerance levels of 2-week-old non-acclimated (NA) and cold-acclimated (CA) Col-0, trx-h2, Trx-h2-V5^{OE}/trx-h2 (lines #1 and #2) (FIGS. 4A-4C), and Trx-h2(C/S)-V5^{OE}/trx-h2(#1 and #2) (FIGS. 4D-4F) plants. FIGS. 4G-4I, Genetic relationship between Trx-h2 and CBFs in cold signaling analyzed by cbfs and Trx-h2-HVE/cbfs (#1 and #2). In FIGS. 4A-4I, after freezing stress, plants were incubated at 4° C. for 12 h under dark and transferred to 22° C. for 5 days before assessing their recovery based on photographs (FIGS. 4A, 4D and 4G), survival rates (FIGS. 4B, 4E and 4H), and electrolyte leakage assay (FIGS. 4C, 4F and 4I). FIG. 4J shows the model elucidating the cold-induced nuclear translocation of Trx-h2 and activation of CBFs. Cold-induced demyristoylation of cytoplasmic Trx-h2 and its subsequent nuclear-translocation reduces all oxidized (inactive) forms of CBFs, including pre-existing CBF oligomers and cold-induced de novo-synthesized monomers. The reduced (active) CBF monomers activate COR genes expression and enhance the freezing tolerance of plants.

FIGS. 5A to 5L show generation of transgenic Arabidopsis lines expressing various forms of CBF1, Trx-h2, and their mutants in trx-h2 and cbfs backgrounds.

FIG. 5A shows DNA constructs used to overexpress CBF1-Myc under the control the CaMV 35S promoter (CBF1-Myc^{OE}) and to express CBF1-Myc under the control of its native promoter (P_{CBF1}) (P_{CBF1}:CBF1-Myc) in Col-0 and trx-h2 backgrounds; both constructs utilized the nopaline synthase (NOS) terminator. FIGS. 5B and 5C show the expression analysis of CBF1 and Trx-h2 in the overexpression lines (CBF1-Myc^{OE}/Col-0 or CBF1-Myc^{OE}/trx-h2) (FIG. 5B) and transgenic lines (P_{CBF1}:CBF1-Myc/Col-0 and P_{CBF1}:CBF1-Myc/trx-h2) (FIG. 5C) by RT-PCR. FIG. 5D shows the Genomic structure of the Arabidopsis T-DNA insertion knockout mutant, trx-h2 (SALK_079507). ATG and TAA represent the start and stop codons, respectively. FIG. 5E shows the confirmation of Trx-h2 knockout in trx-h2 plant by PCR-based genotyping. FIG. 5F shows the schematic diagrams of DNA constructs used for the overexpression of V5-tag fusions of Trx-h2, Trx-h2(G/A), and Trx-h2(C/S) under the control of the CaMV 35S promoter and octopine synthase (OCS) terminator. Agrobacterium tumefaciens strain GV3101 carrying each construct was transformed into the trx-h2 mutant to generate Trx-h2-V5^{OE}/trx-h2, Trx-h2(G/A)-V5^{OE}/trx-h2, and Trx-h2(C/S)-V5^{OE}/trx-h2 overexpression lines. FIGS. 5G and 5H show the expression levels of Trx-h2 mRNA (FIG. 5G) and Trx-h2 protein (FIG. 5H) in various transgenic Arabidopsis analyzed by RT-PCR and western blotting. To quantitatively compare the expression levels of Trx-h2 mRNA and the corresponding protein among various genotypes, two different blots with short (middle panel) and long (upper panel) exposure times in the ChemiDoc™ MP System were shown. FIGS. 5I-5L show DNA constructs for the preparation of cbfs-null variants by CRISPR-cas9 system (FIG. 5I) and Trx-h2-HA^{OE}/cbfs (FIG. 5J) Arabidopsis. Expression levels of Trx-h2 mRNA (FIG. 5K) and Trx-h2 protein (FIG. 5L) in Trx-h2-HA^{OE}/cbfs (lines #1 and #2) plants were analyzed by RT-PCR and western blotting, respectively. In FIGS. 5G-5L, Tubulin and Rbc L were used as loading controls.

FIGS. 6A to 6D show amino acid sequence characteristics of Trx-h2 and its specific interaction with CBF1 at low temperature.

FIG. 6A show the comparison of N-terminal amino acid sequences (˜80 residues) of 11 cytoplasmic Trx-hs in Arabidopsis, and their putative acylation sites predicted by the TermiNator program. Based on the modification pattern of fatty acids, yellow, magenta, green, and cyan boxes on the left represent subgroup I, Sub-II, Sub-III, and Sub-IV Trx-hs, respectively. Critical amino acid residues required for the myristoylation, N-a-acetylation, and palmitoylation of Trx-hs are outlined by a red box (Gly² of Sub-II), blue box (Alae of Sub-I), and green box (Cys⁵ of Sub-III & IV), respectively. The active site Cys residues (CXXC motif) are outlined by a maroon box. FIG. 6B shows the schematic representation of Trx-h2 and its point mutation variants, Trx-h2(G/A) and Trx-h2(C/S). FIG. 6C shows that the sequence features of Trx-h2. Active site Cys residues of Trx-h2 (at amino acid positions 59 and 62) are indicated in bold blue font, and the Gly² residue required for myristoylation (G²) is indicated in green. The conserved Trx motif (122 amino acids) is 15 indicated in red. The bipartite nuclear localization signal (NLS) sequence identified from the NLS-mapper program is underlined. The asterisk at the end of the protein sequence indicates the stop codon. FIG. 6D shows the interacting specificity of CBF1 with Trx-h2, but not with Trx-h3 (control), analyzed by BiFC at 4° C.

FIGS. 7A to 7C show analysis of anti-Trx-h2 antibody specificity, Trx-h2 gene expression and Trx-h2 protein abundance in Col-0 plants at low temperatures.

FIG. 7A shows the specificity of anti-Trx-h2 antibody prepared in our laboratory. Total protein extracts of Col-0, trx-h2, and Trx-h2-V5^{OE}/trx-h2 plants were separated by SDS-PAGE on a 12% polyacrylamide gel and subjected to western blotting using anti-Trx-h2 antibody. Rbc L stained with Ponceau S was used as loading controls. FIG. 7B shows the expression level of Trx-h2 gene transcripts analyzed by qRT-PCR at 4° C. FIG. 7C, Expression level of Trx-h2 protein analyzed by western blot in Col-0 plants at 4° C.

FIG. 8 shows protocol used for the detection of myristoylated Trx-h2 in Arabidopsis. Azidomyristate was vacuum-infiltrated into 2-week-old Trx-h2-V5^{OE}/trx-h2 and Trx-h2(G/A)-V5^{OE}/trx-h2 plants incubated at 22° C. for 24 h. Total proteins extracted from these plants were incubated with phosphine-PEGS-biotin. The biotinyl-myristoylated Trx-h2-V5 was immunoprecipitated with anti-V5 antibody and separated by SDS-PAGE on a reducing gel. Then, the biotinyl-myristoylated Trx-h2-V5 was detected by western blot analysis using anti-biotin antibody. The protocol was established by modifying the method used for the detection of myristoyl proteins in cell cultures.

FIGS. 9A and 9B show subcellular localization of Trx-h2 in Arabidopsis protoplasts at warm temperature.

FIG. 9A shows the schematic diagram of the construct used to express Trx-h2-YFP under the control of the CaMV 35S promoter in Arabidopsis protoplasts. FIG. 9B shows the subcellular location of Trx-h2 in Arabidopsis protoplasts at warm temperature (22° C.). YFP signal was detected by confocal microscopy. Other fluorescent makers including mCherry-HDEL, soybean a-1,2-mannosidase 1-RFP, and NLS-RFP were used to label the endoplasmic reticulum (ER), Golgi complex, and nucleus, respectively, in the second lane (RFP). The panel labeled ‘Merge’ represents overlapped images of YFP and RFP signals. Bright field images are presented in the panel labeled ‘Bright’. Scale bars=20 μm.

FIGS. 10A to 10D show amino acid sequence characteristics of CBFs containing five conserved Cys residues and the effect of Trx-h2 on their protein structures at warm temperatures.

FIG. 10A shows the alignment of the amino acid sequences of CBF1-3 in Arabidopsis. Five conserved Cys residues (at amino acid positions 23, 30, 100, 117, and 137) are outlined in red boxes. Numbers on the right hand side indicate amino acid positions. FIGS. 10B and 10C show the domain structures of CBF1 (FIG. 10B) and CBF1(C/S) (FIG. 10C); in CBF1(C/S), five conserved Cys residues were replaced by Ser residues. Red box at the N-terminus indicates the nuclear localization signal (NLS); green box indicates the AP2 domain; blue box indicates the activation domain. Numbers listed below indicate the amino acid positions. FIG. 10D shows the effect of Trx-h2 on CBFs protein structures analyzed by western blotting on reducing (lower panel) and non-reducing (upper panel) SDS-PAGE gels in various Arabidopsis genotypes at warm temperature (22° C.). ‘O’ and ‘M’ indicate CBF oligomers and monomers, respectively.

FIGS. 11A to 11G show effect of Trx-h2 on the binding of CBF1 to the COR15a promoter and on the expression of CBF mRNAs and proteins at 4° C.

FIG. 11A shows the nucleotide sequence of the COR15a promoter containing two CRT/DRE core motifs at nucleotide positions −439 to −444 (blue box) and −267 to −262 (green box) upstream of the transcription start site (+1 bp). FIG. 11B shows the schematic representation of the COR15a promoter containing two CRT/DRE cis-elements indicated as blue and green boxes. FIG. 11C, Oligonucleotide sequence of biotin-labeled EMSA probe (−275 to −253 bp). FIG. 11D shows the schematic representation of effector, internal control, and reporter constructs used in the luciferase (LUC) assay. FIG. 11E shows the comparison of LUC activity measured in Nicotiana benthamiana leaves expressing P_{35S}:CBF1 under oxidizing (X/XO) or reducing (GSH) conditions at 22° C. FIG. 11F shows the comparison of LUC activity in N. benthamiana leaves expressing P_{35S}:Trx-h2 or P_{35S}: Trx-h2(C/S) incubated at 22° C. or 4° C. FIG. 11G shows the analysis of CBF1-3 transcript levels in Col-0, trx-h2, and trx-h3 (control) plants incubated at 4° C. by qRT-PCR. Data are expressed as mean±SE (n=3 biologically independent samples).

FIGS. 12A to 12C show that Trx-h2 enhances the freezing tolerance of Arabidopsis plants grown in soil through CBF signaling.

FIGS. 12A-12C show the comparison of freezing tolerance among plants of various Arabidopsis genotypes based on their recovery (FIG. 12A), survival rate (FIG. 12B), and electrolyte leakage (%) (FIG. 12C) after the freezing test. To prepare non-acclimated (NA) and cold-acclimated (CA) plants, 18-day-old plants grown in soil at 22° C. were placed in a freezing chamber cooled from 0° C. to the desired temperature at a rate of 2° C. decline per 30 min using a gradient cooling system. The desired temperature was held for 1 h. Then, plants were incubated at 4° C. for 12 h. The CA plants were pre-incubated at 4° C. for 5 days before the freezing test. Genetic relationship between Trx-h2 and CBFs in cold signaling was analyzed using cbfs mutant and Trx-h2-HA^{OE}/cbfs (#1 and #2) plants.

### DETAILED DESCRIPTION

Activities of cold-responsive C-repeat-binding transcription factors (CBFs) are tightly controlled since they not only induce cold tolerance(resistance) but also regulate normal plant growth under temperate conditions. Thioredoxin h2 (Trx-h2), a cytosolic redox protein identified as an interacting partner of CBF1, is normally anchored to cytoplasmic endomembranes via myristoylation at the second glycine residue.

However, upon cold exposure, the demyristoylated Trx-h2 is translocated to the nucleus, where it reduces the oxidized (inactive) CBF oligomers and monomers. The reduced (active) monomers activate cold-regulated genes expression.

Thus, unlike the Arabidopsis trx-h2 null(knock-out) mutant, Trx-h2 overexpression lines were highly cold tolerant. Our findings reveal the mechanism by which cold-mediated redox changes induce the structural switching and functional activation of CBFs, thus conferring plant cold tolerance.

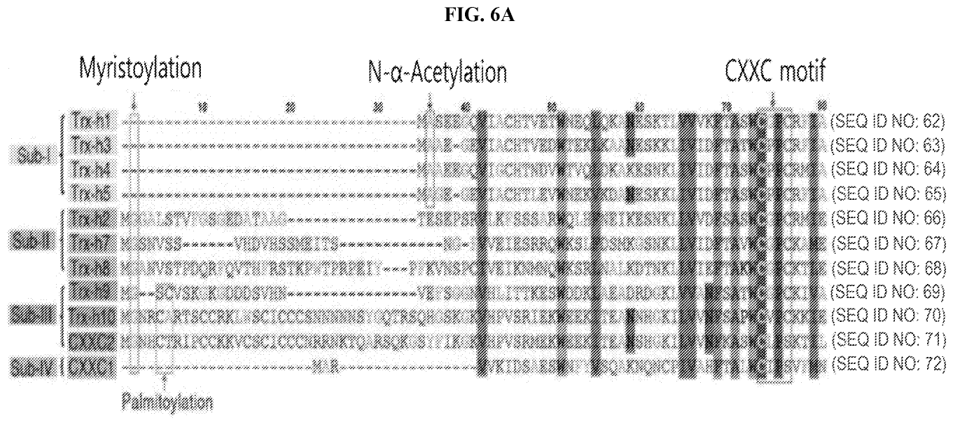

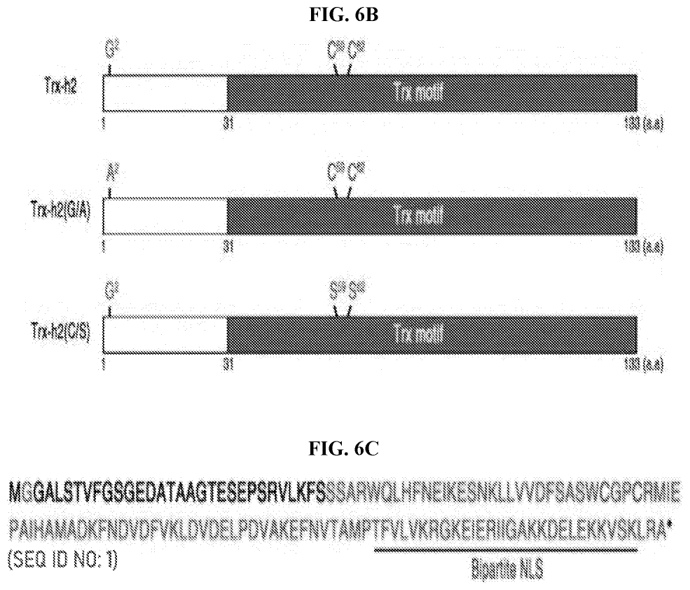

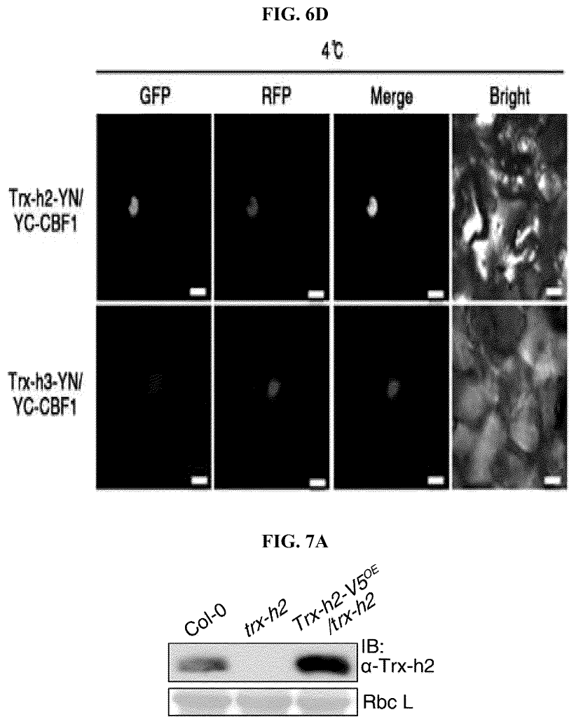

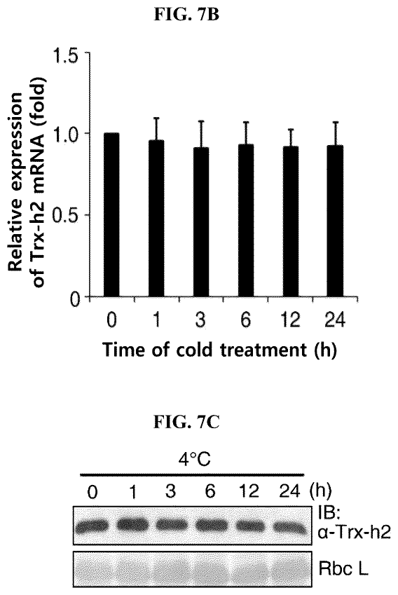

Hereinafter, the present invention will be described in more detail. Among the interactors identified, we selected the h2-type of thioredoxin (Trx-h2), a cytoplasmic redox protein belonging to a subgroup II of Trx-hs (FIGS. 6A to 6C). Trx-h2 contains two active site cysteine (Cys) residues and a putative bipartite nuclear localization signal (NLS) at the C-terminus, predicted by the NLS-mapper program. Trx-h2 is known to sense cellular levels of reactive oxygen species (ROS) and transduce the signal to downstream target proteins. To understand the cold-responsive function of Trx-h2, we investigated its mRNA and protein levels in cold-treated or -untreated Col-0 plants by quantitative real-time PCR (qRT-PCR) and western blot analysis with anti-Trx-h2 antibody (FIGS. 7A to 7C). No difference was detected in Trx-h2 levels between warm and cold conditions, indicating that Trx-h2 possibly regulates CBF activity and protein structure at the post-translational level.

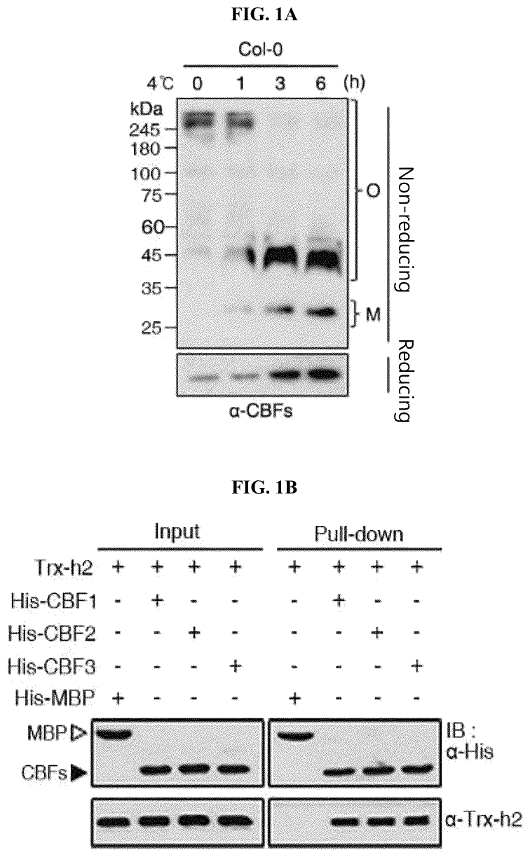

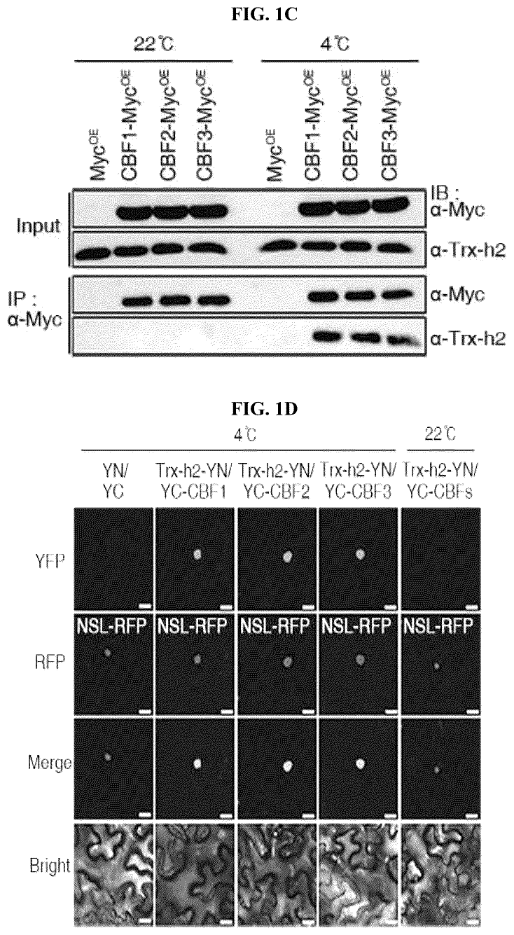

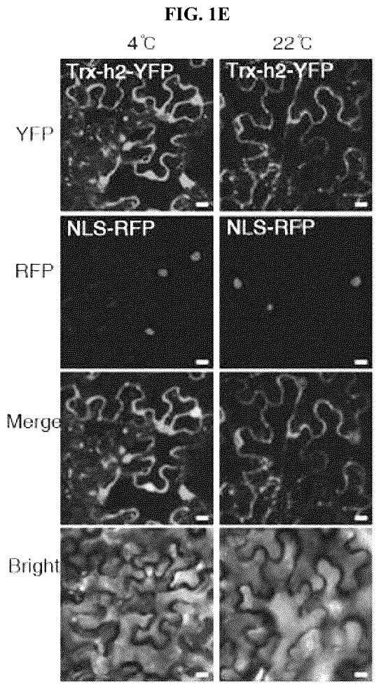

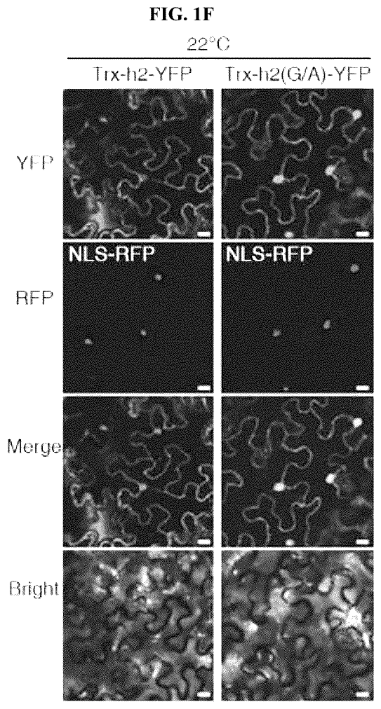

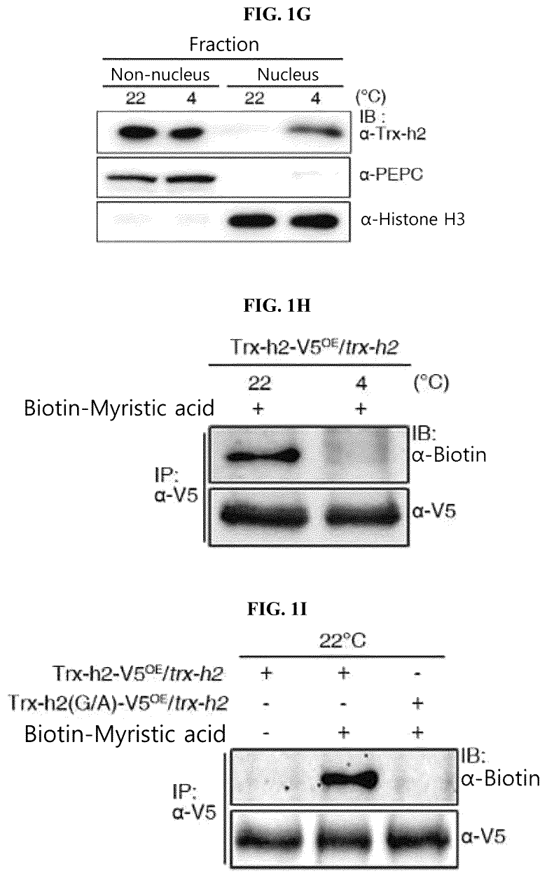

We confirmed the interaction between cytosolic Trx-h2 and nuclear CBFs by in vitro pull-down assay using purified recombinant Trx-h2 and histidine (His)-tagged CBFs (FIG. 1B). Same results were obtained not only in the in vivo IP-assay, using CBFs-Myc^{OE} Arabidopsis incubated for 6 hours at 22° C. or 4° C. (FIG. 1C), in which Trx-h2 fused to the N-terminal half of yellow fluorescent protein (YFP) gene (Trx-h2-YN) was co-expressed with CBFs fused to C-terminal half of YFP (YC-CBFs) in Nicotiana benthamiana leaves (FIG. 1D).

However, Trx-h3 (control) did not interact with CBF in the BiFC assay (FIG. 6D), suggesting that Trx-h2 specifically interacts with CBFs in the nucleus at 4° C. but not at 22° C.

To enable the interaction between cytoplasmic Trx-h2 and nuclear-localized CBFs, Trx-h2 should be transported to the nucleus at low temperature; this was shown by transient expression of Trx-h2-YFP in tobacco leaves (FIG. 1E). The Trx-h2-YFP signal largely overlapped with the NLS-fused red fluorescent protein (RFP) signal at 4° C., but the YFP signal was detected only in the cytosol at 22° C., indicating that cold triggers the translocation of cytoplasmic Trx-h2 to the nucleus.

This was confirmed again by the subcellular fractionation and western blotting of proteins extracted from Col-0 plants (FIG. 1G). Since the three Arabidopsis CBFs (CBF1-3) were indistinguishable in terms of their affinity for Trx-h2 and their molecular properties regulated by Trx-h2, we selected CBF1 as a representative CBF for further analysis.

To examine the molecular mechanism of cold-induced nuclear translocation of Trx-h2, we analyzed its amino acid sequence using the Myristoylator program. The results showed that Trx-h2 contains the second glycine residue (Gly2) conserved in the subgroup II of Trx-hs, a canonical myristoylation site (FIGS. 6A and 6B). Since myristoylation affects the membrane-anchoring efficacy and cellular localization of Trx-h2, we examined the importance of Gly2 for the subcellular localization of Trx-h2 by expressing YFP fusions of Trx-h2 and Trx-h2(G/A) (in which the Gly2 was replaced by Ala) in tobacco leaves (FIG. 1F). The Trx-h2-YFP signal was detected predominantly in the cytoplasm at 22° C.; however, a substantial amount of the Trx-h2(G/A)-YFP signal was detected in the nucleus.

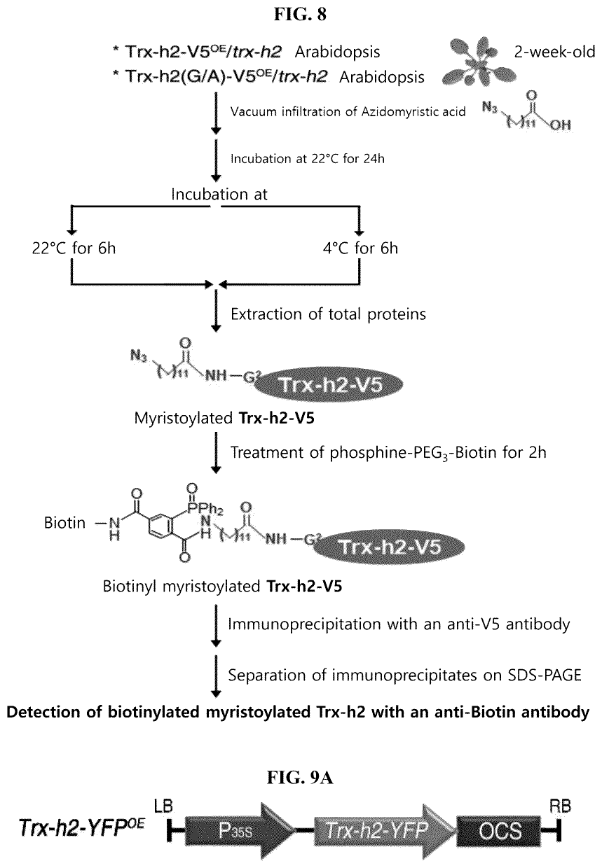

Moreover, to detect the myristate of Trx-h2, Trx-h2-V5^{OE}/trx-h2 and Trx-h2(G/A)-V5^{OE}/trx-h2 plants were vacuum infiltrated with azidomyristate and incubated at different temperatures (FIG. 8). Then the biotinylated azidomyristoylated Trx-h2-V5 was immunoprecipitated by anti-V5 antibody conjugated to protein-G agarose beads and detected with anti-biotin antibody. The biotinylated myristate was detected from Trx-h2-V5^{OE}/trx-h2 plants incubated at 22° C. (FIG. 1H) but not from Trx-h2(G/A)-V5^{OE}/trx-h2 plants (FIG. 1I).

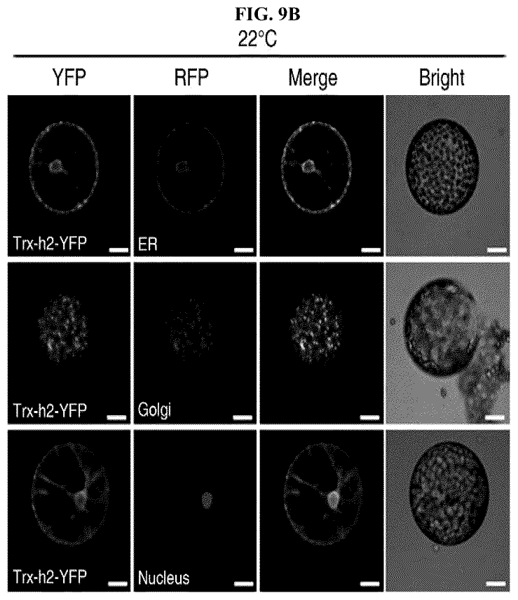

These results suggest that myristate attached to Gly2 anchors Trx-h2 to the cytoplasmic endomembranes at 22° C., whose localization was verified using Arabidopsis protoplasts (FIGS. 9A and 9B). However, the fatty acid is removed by a certain cold-responsive deacylase at 4° C., enabling the demyristoylated Trx-h2 to translocate to the nucleus.

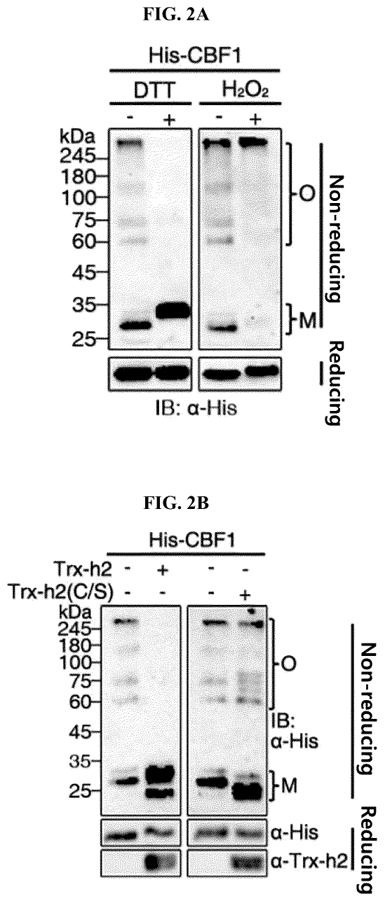

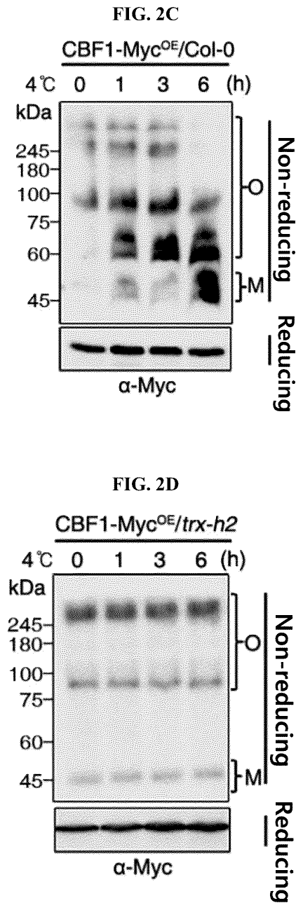

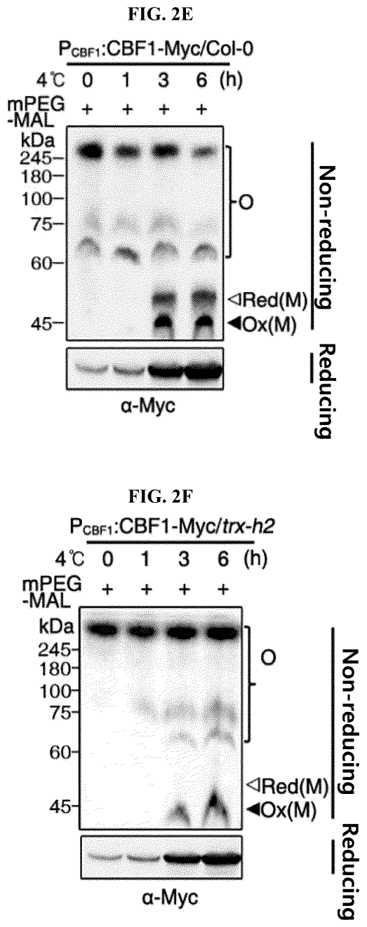

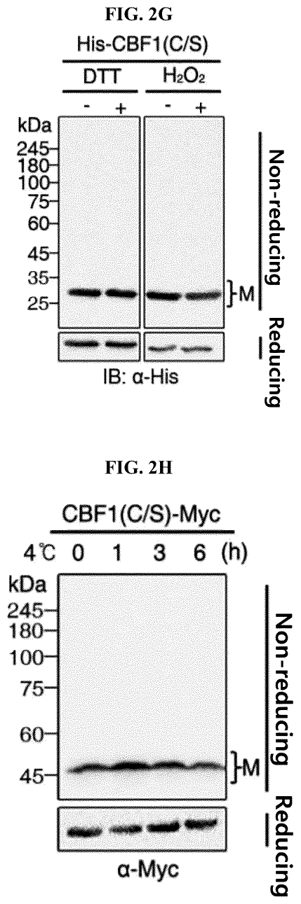

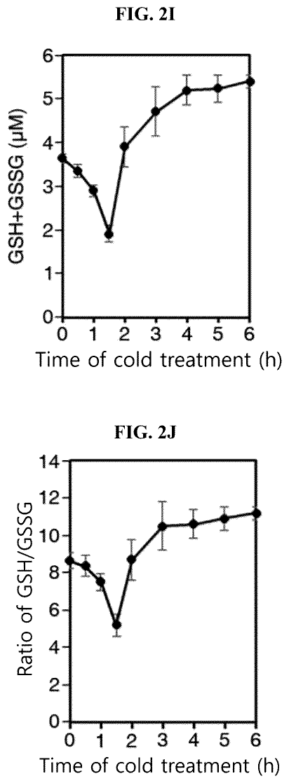

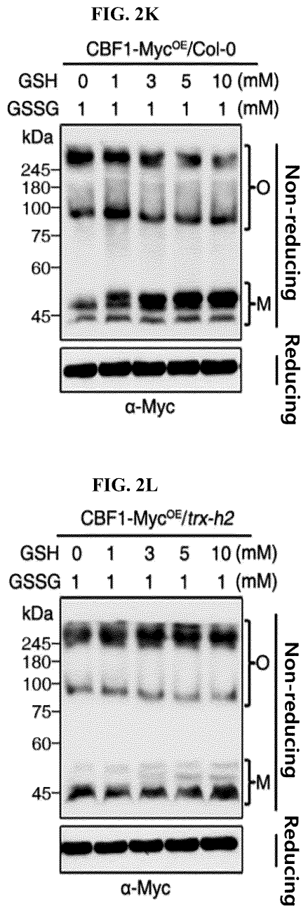

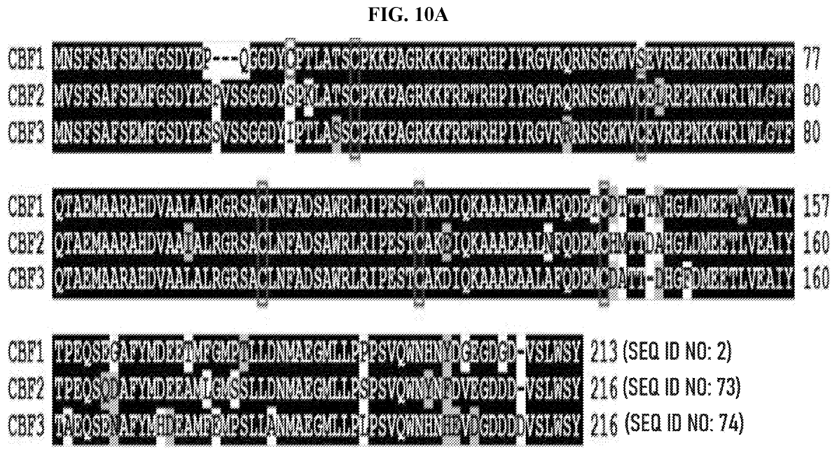

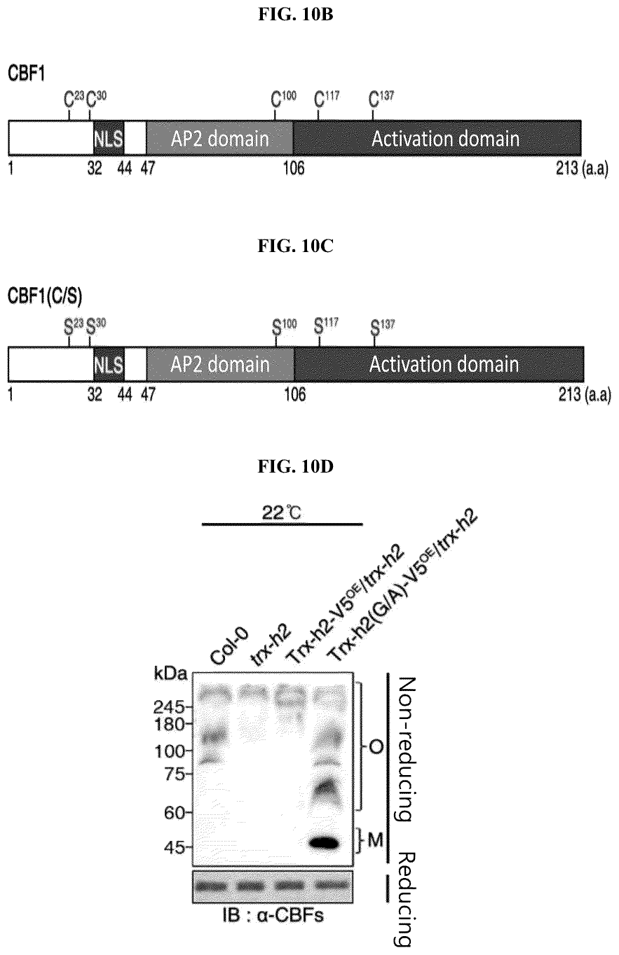

Since several redox regulatory proteins undergo redox-dependent structural modifications and all Arabidopsis CBFs contain five conserved Cys residues (FIGS. 10A to 10D), we analyzed the protein structure of recombinant His-CBF1 at 22° C. in vitro. While CBF1 showed several discretely-sized oligomers on a non-reducing gel (FIG. 2A; lane1), dithiothreitol (DTT) dissociated the oligomers into monomers (lane 2), suggesting that CBF1 oligomers are formed by intermolecular disulfide bonds. Conversely, H₂O₂ induced the formation of HMW oligomers from the middle-sized CBF1 oligomers and monomer (lane 4).

Similarly, the Trx system comprising NADPH, Trx reductase, and Trx-h2 (as an in vivo electron donor) reduced and dissociated CBF1 oligomers into monomers (FIG. 2B; left panel), unlike the Trx system comprising the redox-insensitive Trx-h2(C/S) (right panel), in which the Cys was replaced by Ser. Trx-h2-dependent structural switching was confirmed in planta by overexpressing CBF1-Myc in Col-0 and trx-h2 backgrounds. CBF1 extracted from CBF1-Myc^{OE}/Col-0 plants grown at 22° C. displayed predominantly HMW oligomers on a non-reducing gel (FIG. 2C; lane 1), which were gradually dissociated into small oligomers and monomers by cold (lanes 2-4). By contrast, no structural change was detected in CBF1 extracted from CBF1-Myc^{OE}/trx-h2 plants (FIG. 2D).

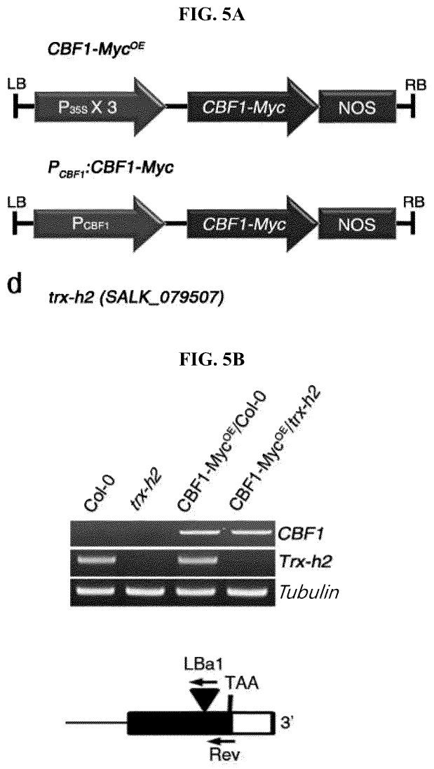

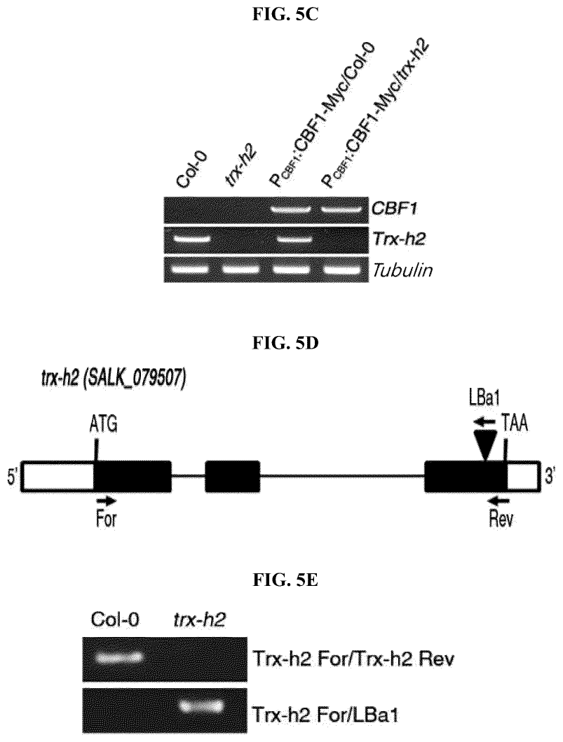

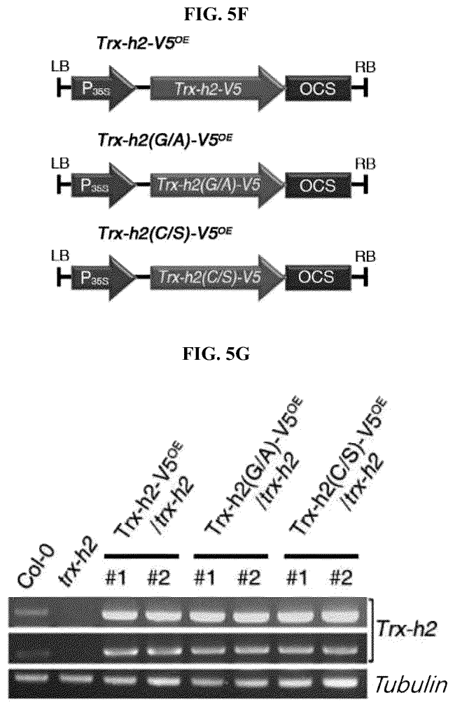

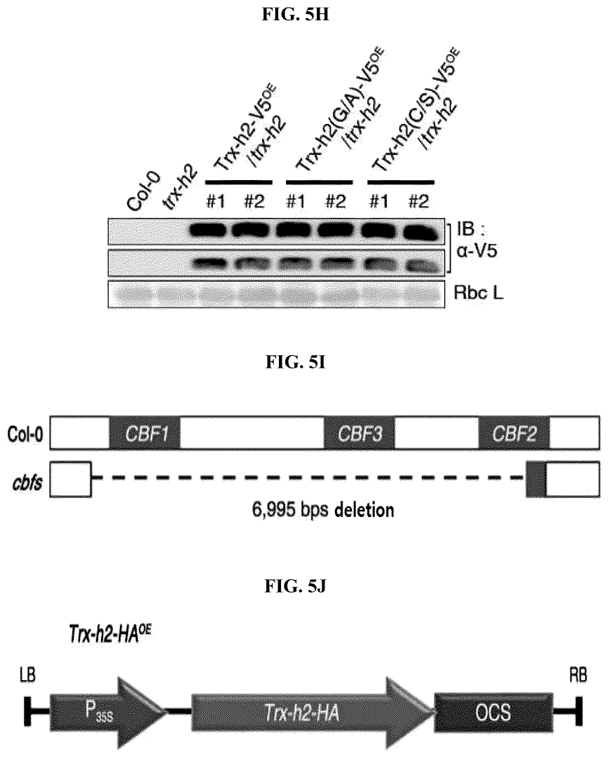

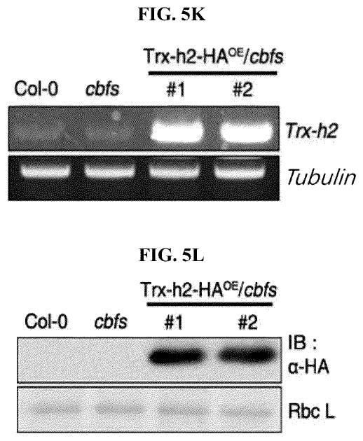

Next, we examined the redox changes of newly-synthesized CBF1 in PcBF1:CBF1-Myc plants upon cold treatment (FIGS. 5A and 5C). After exposing the plants to cold, total proteins were extracted and treated with thiol-alkylating reagent, MM(PEG)24-methyl-PEG-maleimide (mPEG-MAL), which binds to the thiol-group of proteins and increases their MW by 1,200 Da/mPEG-MAL.

In P_{CBF1}:CBF1-Myc/Col-0 plants, the amount of HMW oligomers of CBF1 was decreased by cold treatment, whereas the quantity of two upper and lower monomeric bands corresponding to the reduced and oxidized CBF1 monomer were increased (FIG. 2E). However, in the absence of Trx-h2, cold increased the amount of only the oxidized, but not the reduced, form of CBF1 monomer, with no structural change in HMW oligomer (FIG. 2F).

Furthermore, the recombinant CBF1(C/S) protein, in which all five Cys residues were replaced by Ser (FIG. 10C), and proteins extracted from tobacco leaves expressing CBF1(C/S)-Myc showed a single band of oxidized monomer (FIGS. 2G and 2H).

These results suggest that Trx-h2 reduces all oxidized forms of CBF1 in plants, including the pre-existing CBF1 oligomer at warm temperature and cold-induced newly-synthesized CBF1 monomer, to produce reduced CBF1 monomers at cold. But, Trx-h2(G/A)^{OE}/trx-h2 plants generate critical levels of CBF1 oligomers and monomer at 22° C. (FIG. 10D), suggesting that the temperature-dependent localization of Trx-h2 is the primary factor for CBF1 reduction and monomerization.

To enable the reduction and structural switching of CBF1, Trx-h2 should exist as a reduced form at low temperature. Therefore, we examined cold-induced redox changes in planta by measuring the concentration of intracellular glutathione pool (GSH+GSSG) and GSH/GSSG ratio (FIGS. 21 and 2J), as these are typical indicators of the cellular redox status. Cold triggered a rapid decrease in the total amount of glutathione and the

GSH/GSSG ratio within 1.5 h.

However, normal levels were instantly restored within 3-4 h after cold treatment, probably because plants rapidly produced large amounts of antioxidant enzymes, which enabled Trx-h2 to maintain its reduced status. The cellular redox changes caused reduction and structural dissociation of CBF1 in CBF1-Myc^{OE}/Col-0 plants, but not in CBF1-Myc^{OE}/trx-h2 plants, under the cold condition (FIGS. 2K and 2L). These data suggest that ROS, through Trx-h2, act as cold signaling molecules via intimate association with antioxidant networks.

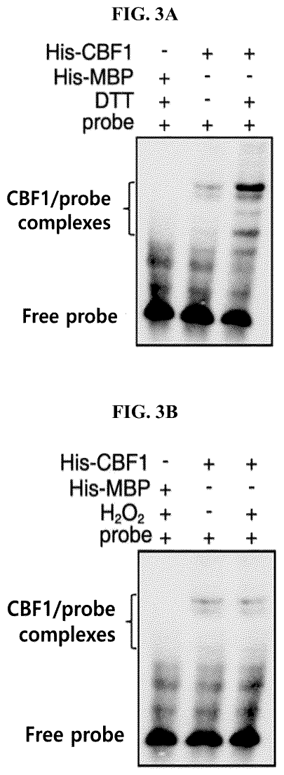

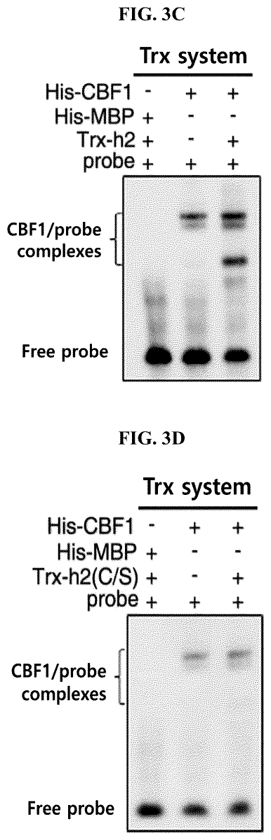

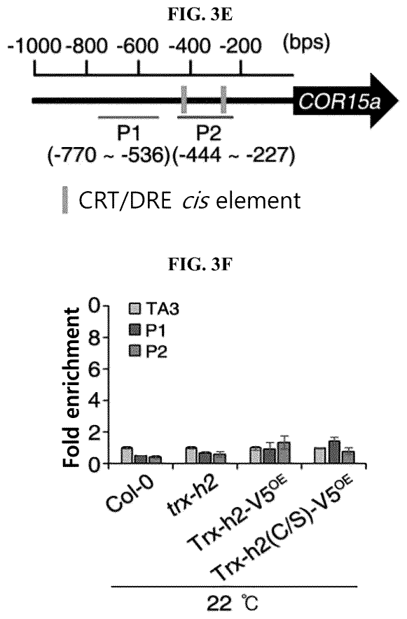

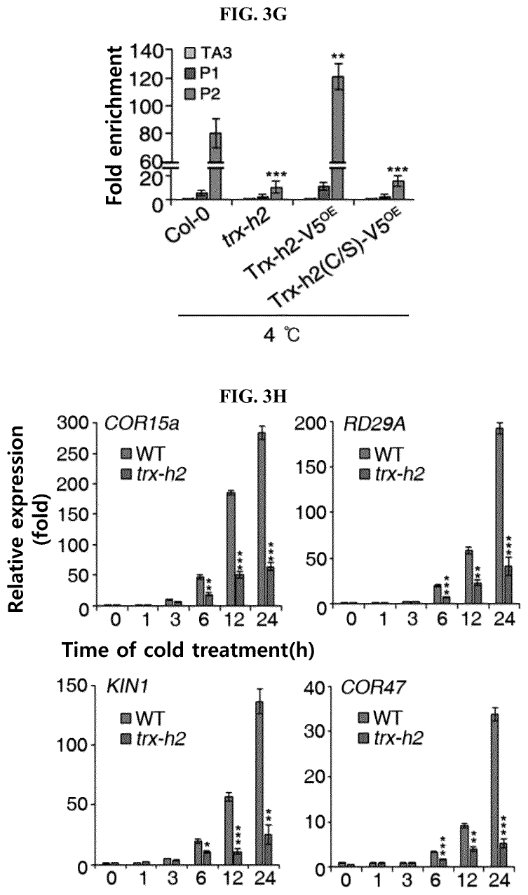

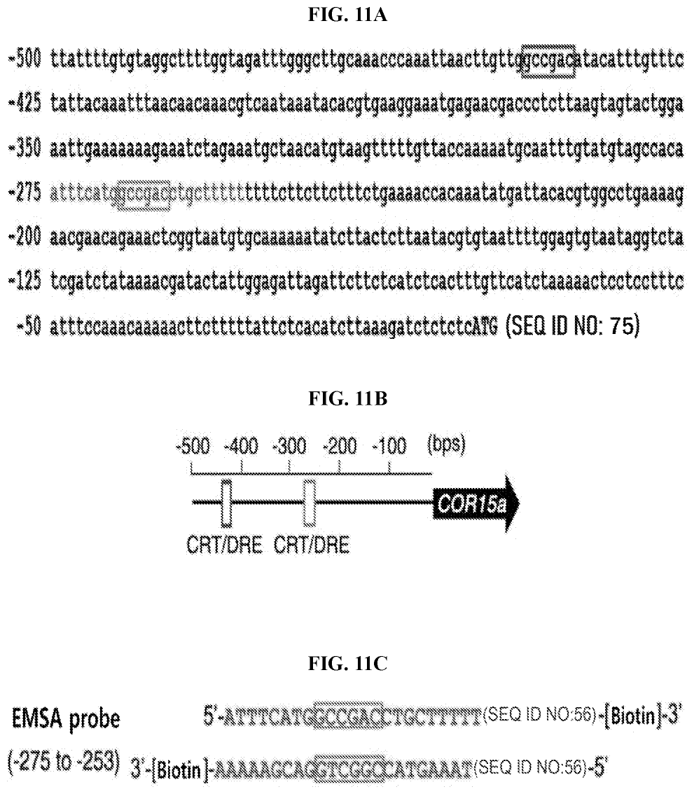

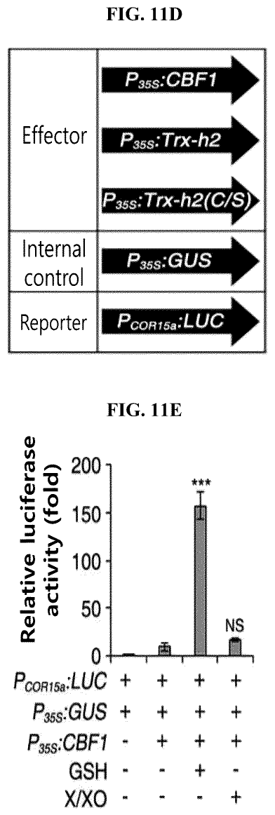

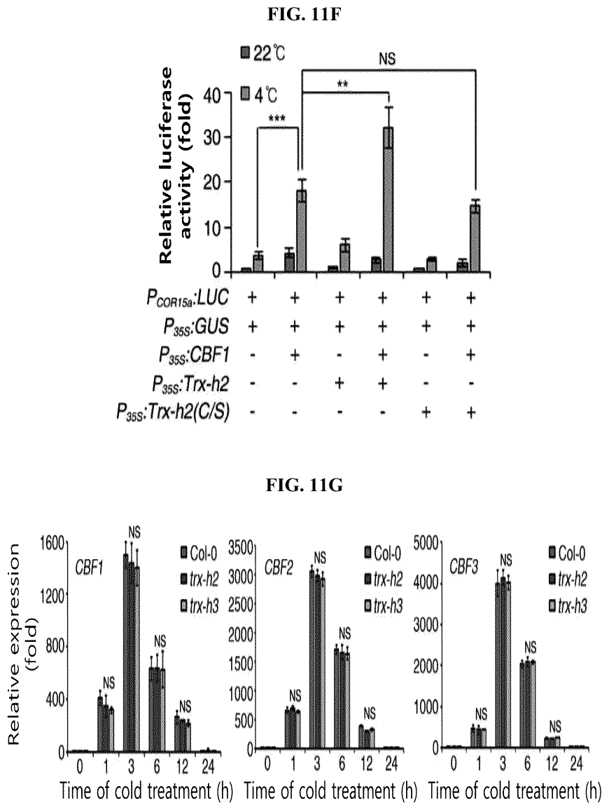

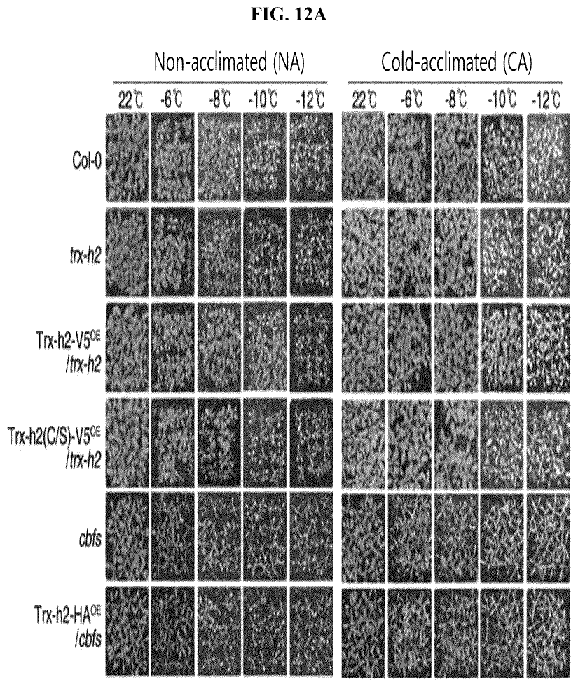

To investigate the physiological significance of Trx-h2-mediated CBF1 reduction and monomerization under cold condition, we performed electrophoretic mobility shift assays (EMSAs) using biotin-labeled oligomer corresponding to the COR15a promoter (a representative CBF1 target gene; FIGS. 11A to 11G) and recombinant proteins of His-CBF1 or His-MBP (control). Treatment with DTT or Trx-h2, but not H₂O₂ or Trx-h2(C/S), induced a distinct mobility shift in the probe interacting with His-CBF1 (FIGS. 3A to 3D).

The binding specificity of CBF1 for P1 and P2 motifs in the COR15a promoter (FIG. 3E) was analyzed by chromatin immunoprecipitation (ChIP) using anti-CBFs antibody and nuclear proteins extracted from various Arabidopsis genotypes (FIGS. 5D to 5H). The co-precipitated DNA fragments were amplified by qRT-PCR using primers specific to P1 and P2 motifs, and the enrichment of COR15a promoter fragments was quantified relative to that of the TA3 promoter (control). While no enrichment was found from the plants incubated at 22° C. (FIG. 3F), the P2 region, containing two CRT/DRE motifs, was significantly amplified compared to the P1 region at 4° C. in Col-0 and Trx-h2-V5^{OE}/trx-h2 plants, but not in Trx-h2(C/S)-V5^{OE}/trx-h2 (FIG. 3G).

Next, we examined whether CBF1 could affect the expression of luciferase (LUC) reporter gene under the control of COR15a promoter in the presence of various effectors (FIG. 11D). LUC activity normalized relative to β-glucuronidase (GUS) activity (internal control) was greatly increased in GSH-treated tobacco leaves transiently expressing CBF1 but not in xanthine/xanthine oxidase (X/XO)-treated leaves.

LUC activity analysis in tobacco leaves expressing Trx-h2 or Trx-h2(C/S) at 22° C. and 4° C. revealed that LUC activity was significantly increased by Trx-h2, but not by Trx-h2(C/S), at 4° C. (FIGS. 11E and 11F). Whereas the expression of all the COR genes was strongly up-regulated in Col-0 and trx-h3 (control) plants at 4° C. (FIG. 3H), however was not up-regulated in trx-h2 plant even though all CBFs in plants were similarly controlled (FIG. 11G). These results indicate that Trx-h2 is essential for the expression of the downstream COR gene by specifically reducing and monomerizing CBF at low temperatures.

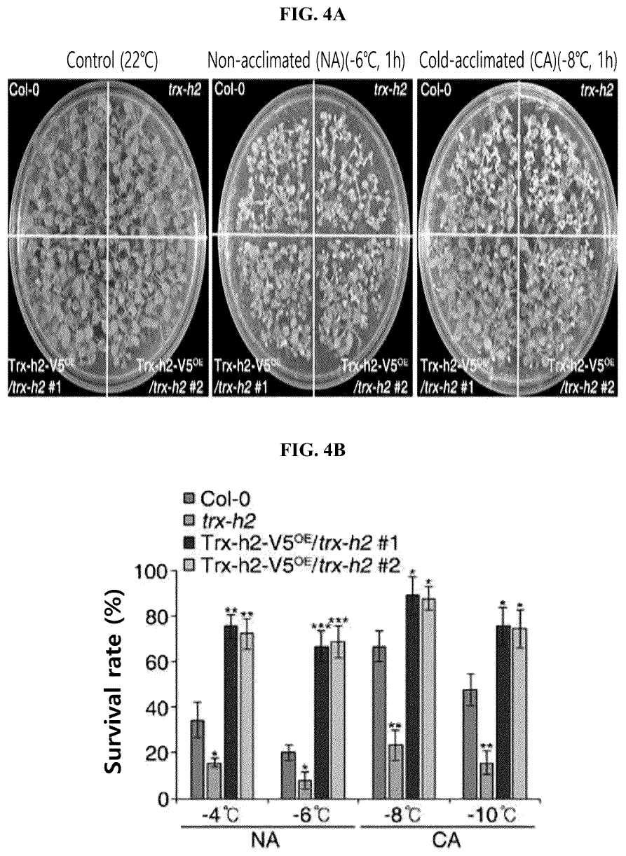

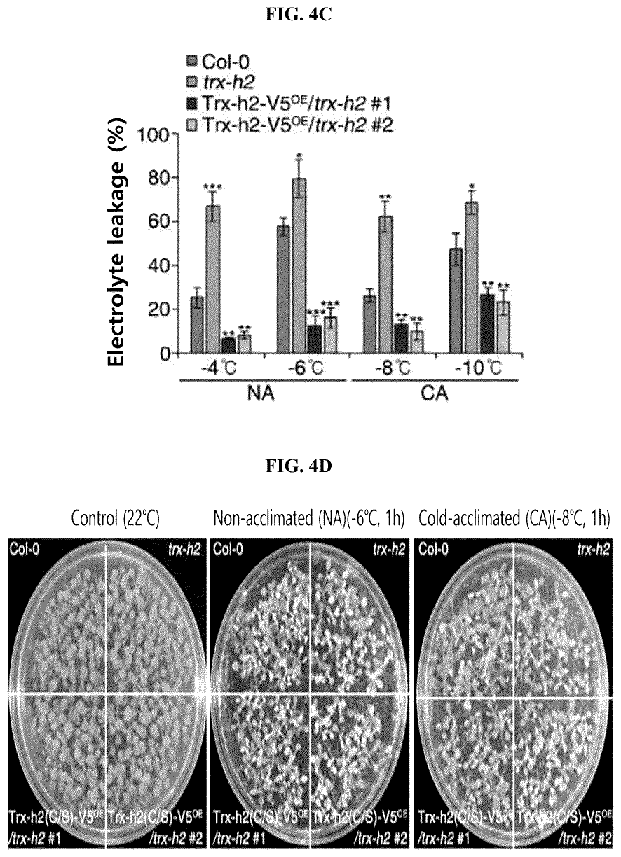

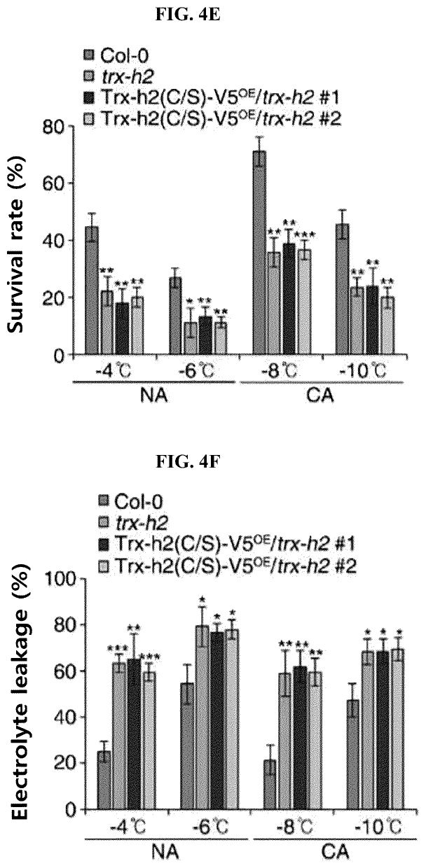

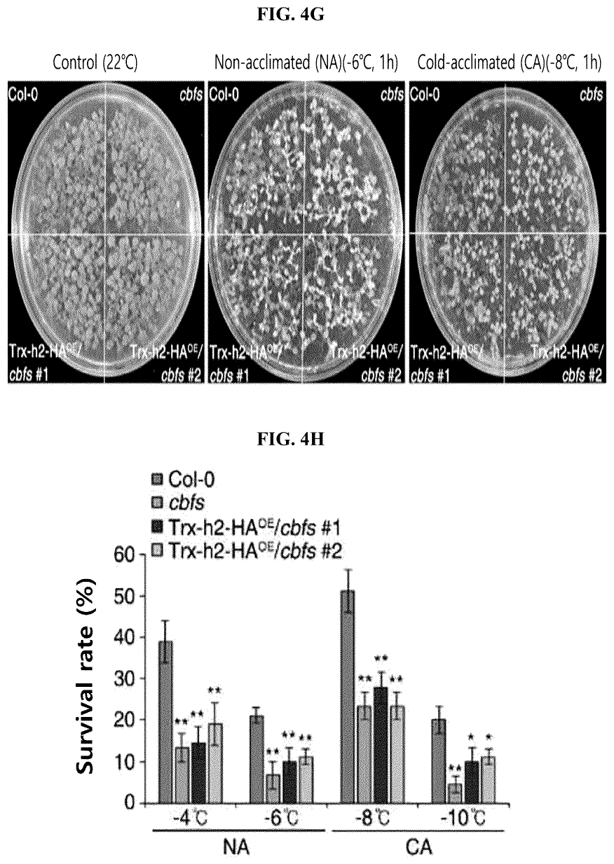

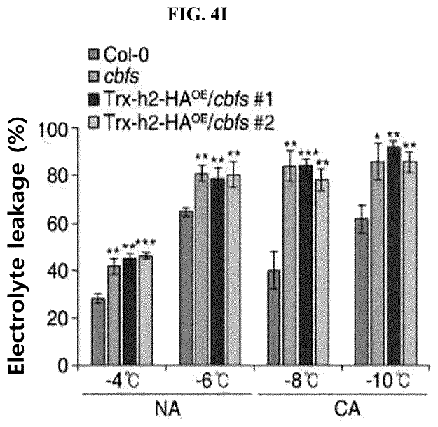

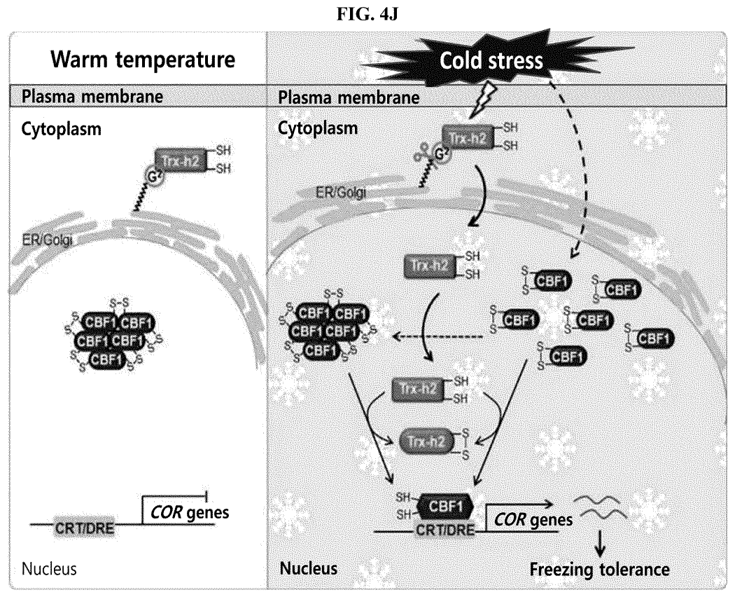

Lastly, we examined the effect of Trx-h2 on freezing tolerance in Arabidopsis genotypes. When non-acclimated (NA) and cold-acclimated (CA) plants grown in agar-media were exposed to freezing temperatures for 1 h, plants of two independent Trx-h2-V5^{OE}/trx-h2 lines (#1 and #2) exhibited greater freezing tolerance, higher survival rates, and lower electrolyte leakage than Col-0 plants (FIGS. 4A to 4C). In contrast, trx-h2 and Trx-h2(C/S)-V5^{OE}/trx-h2 plants showed freezing sensitive phenotypes (FIGS. 4D to 4F).

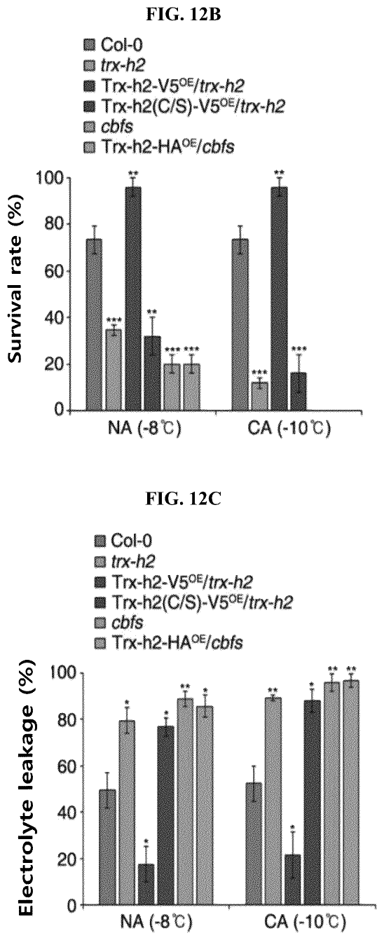

Similarly, NA- and CA-plants of Trx-h2-V5^{OE}/trx-h2 grown in soil, but not the trx-h2 and Trx-h2(C/S)-V5^{OE}/trx-h2 plants, showed freezing tolerance (FIGS. 12A to 12C). To confirm the genetic linkage between Trx-h2 and CBFs, we overexpressed Trx-h2-HA in cbfs background to produce Trx-h2-HVE/cbfs plants and confirmed the lines by analyzing their mRNA and protein levels (FIGS. 51 to 5L). The freezing tolerance, survival rate, and ion-leakage of Trx-h2-HVE/cbfs were similar to those of cbfs plants (FIGS. 4G to 4I and FIGS. 12A to 12C), indicating that the major function of Trx-h2 is genetically linked with CBFs in cold signaling.

The ability of plants to survive from the rapid downturns in temperature majorly depends on the fast response against temperature changes and activation of defense signaling. Our results explicitly demonstrate that cold-induced demyristoylation and release of Trx-h2 from the cytoplasmic endomembranes allows its translocation to the nucleus. The nuclear-localized Trx-h2 reduces and monomerizes all inactive forms of oxidized CBFs, which activate COR genes expression and increase the freezing tolerance of plants (FIG. 4J).

Consequently, a bit of oxidized (inactive) CBF oligomers existed at warm temperature serve as a reservoir that can be quickly activated by cold-induced reduction and monomerization, which initiates COR genes expression prior to cold-mediated CBFs expression and translation. Also, given that cbfs Arabidopsis exhibit germination reduction, dwarfism and stress sensitivity at warm temperature, our results suggest that It was confirmed that oxidized CBF oligomers play an important role in regulating plant growth and development through interactive partner exchange under normal conditions. Redox-mediated regulation and structural switching of cellular crucial proteins are highly sensitive and rapid processes that appear to be conserved across plant and animal kingdoms. For instance, the cytosolic non-expressor of PR1 (NPR1) oligomer in plants is reduced by Trx-h5 upon pathogenic attack and alters its structure to enforce nuclear-translocation and activate immune-responsive genes.

Similarly, the DNA binding and transactivation capacities of the mammalian nuclear activator-protein 1 and nuclear factor-κB, involved in numerous metabolic processes, are regulated by the redox-triggered nuclear-translocation of Trx.

In summary, our results show a delicate framework of redox-dependent structural/functional regulation of CBFs, which represents a very early response of plants to a cold snap. Manipulating the ability of plants to induce low-temperature acclimation could promote the development of varieties with improved freezing resistance and improved crop yields.

Hereinafter, the present invention will be described in more detail with reference to Examples.

### EXAMPLE 1

### Plant Materials and Growth Conditions

Arabidopsis thaliana ecotype Columbia (Col-0) was used as the wild type and for generating transgenic lines overexpressing Trx-h2, Trx-h2(C/S), and Trx-h2(G/A). The T-DNA insertion knockout mutant, trx-h2 (SALK_079507; Col-0 background), was obtained from the Arabidopsis Biological Resource Center (ABRC; Ohio State University, OH, USA). Plants of all genotypes were grown on Murashige and Skoog (MS) medium containing 3% sucrose and 0.25% phyta-gel (pH 5.8) at 22° C. under 16-h light/8-h dark photoperiod. Nicotiana benthamiana plants were grown at 26° C. under 100 μμE·m⁻² s⁻¹ light intensity.

### EXAMPLE 2

### Expression and Purification of Trx-h2 and CBF Recombinant Proteins

Full-length cDNAs of the wild type or mutant Trx-h2 and CBF1-3 genes were inserted into the pET28a expression vector, transformed into Escherichia coli BL21 (DE3) pLysS cells, and cultured in Luria-Bertani (LB) medium at 37° C. After the culture reached an optical density of 0.5-0.6 at 600 nm, protein expression was induced by adding isopropyl-β-D-thiogalactopyranoside (IPTG) to a final concentration of 1 mM. The culture was then grown at 30° C. for 5 h.

Cells were harvested by centrifugation and resuspended in a buffer containing 1.8 mM KH₂PO₄ (pH 8.0), 140 mM NaCl, 2.7 mM KCl, and 10 mM Na₂HPO₄. After disrupting the cells by sonication, proteins were purified using Ni-NTA agarose gel (Incospharm, Daejeon, Korea). His-tagged CBF (His-CBF) was eluted with 100 mM imidazole, and His-fused Trx-h2 was eluted by thrombin cleavage. Recombinant proteins were dialyzed against 20 mM HEPES-NaOH (pH 8.0) buffer and used for biochemical analyses.

### EXAMPLE 3

### Identification of Homozygous trx-h2 Mutant Lines, and Generation of Transgenic Arabidopsis Plants Overexpressing Trx-h2, Trx-h2(C/S), and Trx-h2(G/A)

Homozygous trx-h2 mutant lines were identified by genotyping. To generate Trx-h2-V5^{OE}/trx-h2, Trx-h2(C/S)-V5^{OE}/trx-h2, Trx-h2(G/A)-V5^{OE}/trx-h2, and Trx-h2-HA^{OE}/cbfs overexpression lines, cDNAs of Trx-h2, Trx-h2(C/S), and Trx-h2(G/A) were amplified using sequence-specific primers (Table 1) and cloned into the pCAMBIA 1300 binary vector. The plasmids were fused with the V5-tag and cloned downstream of the cauliflower mosaic virus (CaMV) 35S promoter in the pEarlyGate301 (pEG301) vector.

The resulting plasmids were introduced into Agrobacterium tumefaciens strain GV3101 and used to transform trx-h2 and cbfs mutant plants via the floral dip method³¹. After selecting T1 transgenic lines on MS plates supplemented with appropriate antibiotics, the expression of transgenes was analyzed by western blotting and reverse transcription PCR (RT-PCR). Homozygous T3 lines were used in all subsequent experiments. CBF1-Myc^{OE}/trxh-2 and P_{CBF1}:CBF1-Myc/trx-h2 plants were generated by crossing homozygous CBF1-Myc^{OE} and P_{CBF1}:CBF1-Myc plants with trxh-2 mutant plants, respectively.

### EXAMPLE 4

### Identification of CBF1-Interacting Proteins by Immunoprecipitation (IP)/Mass Spectrometry Assays

To identify the interacting partners of CBF1 in Arabidopsis, total proteins (100 mg) were extracted from 2-week-old CBF1-Myc^{OE} seedlings exposed to 4° C. for 6 h, and immunoprecipitated using an anti-Myc agarose (Sigma-Aldrich, St. Louis, MO, USA). The CBF1-My c-interacting proteins were washed three times at 4° C. with a buffer containing 50 mM Tris-HCl (pH 7.5), 150 mM NaCl, 1 mM EDTA, 1 mM DTT, and 0.1% NP40. The purified proteins were mixed with SDS-PAGE loading dye, heated at 90° C. for 5 min, and separated by SDS-PAGE. Proteins were identified by matrix assisted laser desorption/ionization-time of flight (MALDI-TOF)/TOF-mass spectrometry.

### EXAMPLE 5

### Detection of the Myristyl Group Attached to Trx-h2

The myristyl (C14) group covalently attached to Trx-h2 in plants was detected using procedures summarized in FIG. 8. This protocol was a modification from the scheme used previously for cell culture proteins. To detect the myristyl group from Trx-h2-V5, 40 μM of azidomyristate (myristic acid, azide; Life Technologies, Camarillo, CA, USA) dissolved in dimethyl sulfoxide (DMSO) was vacuum-infiltrated into 2-week-old Trx-h2-V5^{OE}/trx-h2 or Trx-h2(G/A)-V5^{OE}/trx-h2 seedlings and incubated at 22° C. for 1 day.

These seedlings were ground in liquid N₂, and total proteins were extracted using the IP buffer. After centrifuging the solution at 12,000×g for 10 min, the supernatant (250 μg protein) was reacted with 250 μM phosphine-PEGS-Biotin at 37° C. for 2 h. Using protein-G agarose beads conjugated to anti-VS antibody (Thermo Fisher Scientific, Rockford, IL, USA), biotinylated proteins were immunoprecipitated at 4° C., separated by SDS-PAGE, and detected by western blotting. Biotinylated azidomyristoylated Trx-h2-V5 was detected using an ECL detection kit with both anti-biotin (Abcam, Cambridge, MA, USA) and anti-VS antibodies using the ChemiDoc™ MP System (BioRad, Munich, Germany).

### EXAMPLE 6

### Analysis of Redox-Dependent Structural Switching of CBF1

Redox-dependent structural modification of CBF1 was examined in vitro using purified recombinant His-CBF1 or His-CBF1(C/S) protein incubated with H₂O₂ (oxidizing agent) or DTT (reducing agent) at 25° C. for 15 min. The effect of the reducing power of Trx-h2 on His-CBF1 was analyzed at 25° C. for 30 min using a Trx system containing NADPH, Trx reductase, and either Trx-h2 or Trx-h2(C/S). Proteins were separated by SDS-PAGE on reducing and non-reducing gels, and protein structures were detected by western blotting with anti-His (Abcam, Cambridge, MA, USA) and anti-Trx-h2 antibodies. The structural switching of CBF1 was also analyzed in vivo using 2-week-old CBF1-Myc^{OE}/Col-0 plants.

The functional role of Cys residues in CBF1 structural changes was analyzed by cloning the mutant CBF1(C/S) gene into the pCAMBIA vector containing the Myc-tag. The resulting pCAMBIA-Myc-CBF1(C/S) plasmid was transformed into A. tumefaciens strain GV3101, which was used to infiltrate the leaves of 4-week-old N. benthamiana plants. These plants were subjected to cold treatment at 4° C. for 1, 3, or 6 h. The infiltrated leaves were frozen in liquid N₂, ground to a fine powder, and used for extracting total proteins. To investigate the in vivo structures of CBF1 and CBF1(C/S), proteins were separated by SDS-PAGE on non-reducing gels and detected by western blotting using anti-Myc antibody.

### EXAMPLE 7

### Thiol-Trapping Assay

The thiol-trapping assay of CBF1 was performed in vivo using the 2-week-old Arabidopsis plants expressing CBF1-Myc under its native promoter in Col-0 or trx-h2 mutant background (P_{CBF1}:CBF1-Myc/Col-0 or P_{CBF1}:CBF1-Myc/trx-h2). Plants grown on MS media were exposed to cold (4° C.) for 1, 3, or 6 h. Plant tissues were then frozen and ground in liquid N₂, and total proteins were extracted from plant tissues using the SDS sample buffer containing 2% SDS, 62.5 mM Tris-HCl (pH 6.8), 7.5% glycerol, 0.01% bromophenol blue, and protease inhibitor cocktail. Protein extracts were incubated with the thiol-labeling reagent, MM(PEG)24-methyl-PEG-Maleimide (mPEG-MAL) (Thermo Fisher Scientific, Rockford, IL, USA), for 1 h and centrifuged at 12,000×g for 10 min. Proteins in the supernatant were separated by SDS-PAGE on non-reducing gels and detected by western blotting using anti-Myc antibody.

### EXAMPLE 8

### Isolation and Quantification of RNAs by Quantitative Real-Time PCR (qRT-PCR)

Total RNAs (2 μg) were isolated from Arabidopsis plants using the RNA purification kit (Macherey-Nagel, Duren, Germany), and cDNA was synthesized using a cDNA synthesis kit (Thermo Fisher Scientific, Rockford, IL, USA). Then, 10 ng of cDNA was used as the template for qRT-PCR under the following conditions: 5 min incubation at 95° C., followed by 25 cycles of 30 s at 95° C., 30 s at 56° C., and 1 min at 72° C. Ubiquitin10 (UBQ10) and Acting (ACT2) genes were used as internal controls. Three independent experiments were performed for each sample using gene-specific primers (Table 1).

### EXAMPLE 9

### Co-Immunoprecipitation (Co-IP) Assay

CBF-Myc^{OE} plants were frozen in liquid N₂ and ground to a fine powder. Total proteins were extracted from the ground tissue using IP buffer containing 50 mM Tris-HCl (pH 7.5), 150 mM NaCl, 1 mM EDTA, 0.5% NP-40, 1 mM PMSF, and protease inhibitor cocktail. The isolated proteins were incubated with anti-Myc agarose beads overnight at 4° C., and then washed with IP buffer three times. From the beads, CBF-Myc-proteins were eluted by heating at 90° C. for 1 min, and the proteins were separated by SDS-PAGE. CBF and Trx-h2 proteins were detected using an ECL detection kit (GE Healthcare Life Science, South Plainfield, NJ, USA) with anti-Myc (Cell Signaling Technology, Beverly, MA, USA) and anti-Trx-h2 antibodies, respectively.

### EXAMPLE 10

### Bimolecular Fluorescence Complementation (BiFC) Assay

Interaction between Trx-h2 and CBFs under specific conditions was analyzed using the BiFC assay. Trx-h2 and CBFs were cloned individually into the gateway BiFC-binary vector, pDONR221, to generate pDONR221: Trx-h2 and pDONR221: CBF plasmids, respectively. The Trx-h2-YN plasmid was generated by fusing Trx-h2 with the N-terminal fragment of YFP (YN), while the YC-CBF plasmids were constructed by fusing each of the three CBFs with the C-terminal fragment of YFP (YC). These plasmids were introduced into A. tumefaciens strain GV3101, and the transformed cells were used to infiltrate the leaves of 4-week-old N. benthamiana plants. These plants were incubated at 26° C. for 2 days and then exposed to 4° C. for 6 h. Fluorescence signals generated by YFP were analyzed under a confocal microscope using the FV10-ASW 3.1 software.

### EXAMPLE 11

### Subcellular Fractionation of Nuclear and Non-Nuclear Proteins

To fractionate nuclear and non-nuclear proteins from Trx-h2-V5^{OE}/trx-h2 plants with or without cold treatment at 4° C. for 6 h, the CelLytic PN Extraction Kit (Sigma-Aldrich, St. Louis, MO, USA) was used according to the manufacturer's instructions. Plant tissues were frozen in liquid N₂, ground to a fine powder, and mixed with 1×Nuclei Isolation Buffer (NIB). Samples were centrifuged at 1,260×g for 10 min, and the supernatant containing the non-nuclear protein fraction was separated from the pellet, which contained the nucleus and other subcellular organelles. The pellet was resuspended in 1×NIB buffer containing 10% Triton X-100 (NIBA). Organelle membranes were lysed by adding 10% Triton X-100 to a final concentration of 0.3%, and the lysates in 1×NIBA buffer were applied to a 1.5 M sucrose cushion. Centrifugation of the solution at 12,000×g for 10 min resulted in a semi-pure preparation of nuclei. The pellet was resuspended in NIB and used as the nuclear fraction. The purity of the nuclear and non-nuclear fractions was confirmed by western blotting using anti-histone H3 (Abcam, Cambridge, MA, USA) and anti-PEPC antibodies (Agrisera, Sweden), representing nuclear and non-nuclear standard markers, respectively.

### EXAMPLE 12

### Measurement of the Total Glutathione Pool (GSH+GSSG) and GSH/GSSG Ratio

Time-dependent redox changes in Col-0 plants under cold stress (at 4° C.) were estimated by measuring the total level of glutathione (GSH+GSSG) and the GSH/GSSG ratio in 2-week-old Arabidopsis plants. Plants were ground in liquid N₂, and 20 mg of the powder was dissolved in 200 μl of lysis buffer containing 2% meta-phosphoric acid and 2 mM EDTA. After centrifuging the solution at 13,000×g for 15 min, the pH of the supernatant was adjusted to 5.6 by adding 10% sodium citrate. Then, the level of GSH in 50 μl of the supernatant was measured by reacting the supernatant with 50 μl of the GSH assay mixture (GAM) containing thiol green indicator (a non-fluorescent dye) for 30 min at 30° C. Fluorescence emitted from the reaction product was measured using GEMINI, XPS-spectrofluorometer (Molecular Devices, San Jose, CA, USA), and GSH was quantified from the GSH standard curve.

In addition, the total level of glutathione (GSH+2GSSG) in 50 μl of the supernatant was measured by reacting the supernatant with 50 μl of the total glutathione assay mixture (TGAM) containing NADPH, GSH-reductase, and thiol green indicator for 30 min at 30° C.; the TGAM converts GSSG to 2GSH. After assessing the total amount of glutathione (GSH) by fluorescence, GSSG level was calculated by subtracting the GSH level measured by the GAM from the total concentration of GSH+GSSG evaluated by TGAM. Average concentrations of GSH and GSSG were determined from at least three independent experiments.

### EXAMPLE 13

### Electrophoretic Mobility Shift Assay (EMSA)

To carry out EMSAs, a biotin-labeled oligonucleotide probe (Table 1) was processed using a lightshift™ chemiluminescent EMSA Kit (Thermo Fisher Scientific, Rockford, IL, USA). Recombinant CBF1 (5 μg) was incubated with 40 fmol of biotin-labeled probe and 20 μl of reaction mixture containing 25 ng/μl Poly (dl-dC) binding buffer at 25° C. for 30 min. The reaction products were resolved on a 6% polyacrylamide gels. Probes bound to CBF1 were transferred to charged Hybond-N membrane and detected by western blotting with anti-biotin antibody (Abcam, Cambridge, MA, USA).

### EXAMPLE 14

### Transactivation Assay of CBF1 in N. benthamiana Leaves

Trx-h2-mediated transactivation of CBF1 was assayed in planta. Cells of A. tumefaciens strain GV3101 carrying the reporter construct (P_{COR15a}:LUC) along with either the effector construct (P_{35S}:CBF1, P_{35S}: Trx-h2, or P_{35S}:Trx-h2(C/S)) or the internal control (P_{35S}: GUS) were infiltrated into the leaves of 4-week-old N. benthamiana plants. After 2 days of incubation, plants were exposed to cold (4° C.) for 24 h. Then, leaf tissues were frozen in liquid N₂ and ground to a fine powder. Total proteins were extracted from the ground tissue using IP buffer, and the extract (100 μg protein/20 μl IP buffer) was mixed with 80 μl of GUS/LUC reaction buffer (50 mM Na₂PO₄ [pH 7.0], 10 mM EDTA, 10 mM β-mercaptoethanol, and 0.1% Triton X-100).

Then, 100 μl of LUC substrate (20 mM Tricine, 2.7 mM MgSO₄, 30 mM DTT, 1 mM luciferin, and 0.5 mM ATP) was added to the solution and incubated for 10 min. LUC activity was measured using the GloMax® 20/20 Luminometer (Promega, Madison, WI 53711 USA). To assay GUS activity, protein extracts were mixed with the GUS/LUC substrate solution containing 16.7% methanol and 1.1 mM 4-methylumbelliferyl-β-D-glucuronide hydrate (MUG), and incubated for 10 min (Sigma-Aldrich, St. Louis, MO, USA). The reaction was stopped by the addition of 130 mM Na₂CO₃, and GUS activity was analyzed by measuring MUG fluorescence with GEMINI, XPS spectrofluorometer at excitation and emission wavelengths of 364 and 447 nm, respectively. LUC activity was normalized relative to GUS activity (internal control), and the relative LUC activity was represented as the average of three independent experiments.

### EXAMPLE 15

### Chromatin Immunoprecipitation (ChIP) Assay

The binding of CBF1 to CRT/DRE motifs in the COR15a promoter was analyzed by ChIP using proteins extracted from 2-week-old Arabidopsis seedlings incubated at 4° C. or 22° C. for 6 h. Plant tissues (3 g) were frozen and ground in liquid N₂. The CBF1-bound chromatin complexes were isolated from the ground tissue using lysis buffer (pH 8.0) containing 60 mM HEPES, 1 M sucrose, 5 mM KCl, 5 mM MgCl₂, 5 mM EDTA, 0.6% Triton X-100, 1 μg/ml pepstatin A, and protease inhibitor cocktail (Mini-complete tablet; Roche, Basel, Switzerland).

After the protein-chromatin complexes were crosslinked by 1% formaldehyde, they were sonicated and pulled down using anti-CBFs antibody or rabbit IgG serum (negative control) and salmon sperm DNA/Protein-A agarose (Millipore, Burlington, MA, USA). The agarose beads were sequentially washed with the following buffer solutions: low salt buffer (150 mM NaCl, 0.1% SDS, 1% Triton X-100, 2 mM EDTA, and 20 mM Tris-HCl [pH 8.0]), high salt buffer (500 mM NaCl, 0.1% SDS, 1% Triton X-100, 2 mM EDTA, and mM Tris-HCl [pH 8.0]), LiCl wash buffer (250 mM LiCl, 1% Nonidet P-40, 1% deoxycholate, 1 mM EDTA, and 10 mM Tris-HCl [pH 8.0]), and lastly TE buffer (10 mM Tris-HCl [pH 8.0] and 1 mM EDTA). The protein—DNA complexes were eluted using the elution buffer containing 1% SDS and 0.1 M NaHCO₃, and proteins were removed by overnight incubation at 65° C. in the presence of 5 M NaCl.

After the residual proteins were degraded by proteinase K, DNAs were purified by phenol/chloroform/isoamyl alcohol extraction and precipitated by ethanol precipitation. The purified DNAs were resuspended in TE buffer (pH 8.0), and enrichment of target DNA fragments was determined by quantitative PCR (qPCR). Relative enrichment of target DNA was calculated first by normalization against the UBQ10 gene (internal control) and then against the corresponding amount of target DNA in the input. Primers used for qPCR are listed in Table 1.

### EXAMPLE 16

### Freezing Tolerance Assay

Freezing tolerance assay for non-acclimated (NA) and cold-acclimated (CA) plants was done at −6° C. and −8° C., respectively, by using 18-day-old Arabidopsis plants grown in soil and 2-week-old plants grown in MS agar medium at 22° C. (control). For CA plants, before freezing stress treatment, cold was pretreated at 4° C. for 5 days. The plants were subjected to a freezing chamber (RuMED4001, Stuttgart, Germany), whose temperature was decreased from 0° C. to the target temperature at a rate of 2° C. per 30 min, and target temperature was held constant for 1 h.

After freezing stress, all the plants were incubated at 4° C. in the dark for 12 h and then at 22° C. in the light for 5 days to monitor recovery.

Freezing tolerance of the various Arabidopsis genotypes was assessed by measuring the morphological phenotypes, survival rate (percent green plants recovered after freezing), and electrolyte leakage (%; measured with the fully developed rosette leaves of 4-week-old plants grown in soil), comparing with those of WT plants. At least three sets of the following experiments were conducted to measure electrolyte leakage in plants and were expressed as an average±standard error (SE). One incised leaflet was placed in a test tube containing 100 μl of distilled water and placed in a circulation tank at 0° C. for 1 hour.

After adding ice crystals to each tube, the tube was incubated in a circulation tank, and its temperature was programmed to decrease at a rate of 2° C. per 30 minutes until the desired temperature was reached. After incubating the tube at the desired temperature for 1 hour, it was immediately moved to an ice bath to allow gradual thawing of the tube. After thawing, all contents in the tube were transferred to a new tube containing 20 ml of distilled water and shaken overnight. The conductivity of the solution was measured using a conductivity meter (Cole-Parmer Instrument, Vernon Hills, IL, USA). Subsequently, the tube containing the leaflet was sterilized at 100° C. for 15 minutes at high pressure, cooled to room temperature in a shaker and the conductivity of the solution was measured again. The electrolyte leakage ratio before and after the autoclave was used as an indicator of membrane damage due to freezing stress.

TABLE 1

| | |
|---|---|
| Primer | Sequence (5′−>3′) |

Primers for constructs for plant transformation

| | |
|---|---|
| Trx-h2 F (ClaI) | ATCGATATGGGAGGAGCTTTATCAACT (SEQ ID NO: 3) |
| Trx-h2 R (XbaI) | TCTAGATGCTCTGAGTTTGCTAACTTTCTT (SEQ ID NO: 4) |
| Trx-h2(C/S) F | GGCCTCATGGTCCGGACCATCTAGGATGA (SEQ ID NO: 5) |
| Trx-h2(C/S) R | TCATCCTAGATGGTCCGGAGCATGAGGCC (SEQ ID NO: 6) |
| Trx-h2(G/A) F | GGATCCATGGCAGGAGCTTTATC (SEQ ID NO: 7) |
| (BamHI) | |

Primers for RT-PCR and genotyping PCR

| | |
|---|---|
| Trx-h2 F | ATGGGAGGAGCTTTATCAAC (SEQ ID NO: 8) |
| Trx-h2 R | TTATGCTCTGAGTTTGCTAA (SEQ ID NO: 9) |
| LBal | TGGTTCACGTAGTGGGCCATCG (SEQ ID NO: 10) |
| Tubulin F | CCAACAACGTGAAATCGACA (SEQ ID NO: 11) |
| Tubulin R | TCTTGGTATTGCTGGTACTC (SEQ ID NO: 12) |

Primers for quantitative real-time PCR

| | |
|---|---|
| Trx-h2 qF | ACAGCTGCAGGGACCGAAT (SEQ ID NO: 13) |
| Trx-h2 qR | CCGAGCGGAAGAGCTAAACTT (SEQ ID NO: 14) |
| UBQ10qF | GGCCTTGTATAATCCCTGATGAATAAG (SEQ ID NO: 15) |
| UBQ10Qr | AAAGAGATAACAGGAACGGAAACATAGT (SEQ ID NO: 16) |
| ACT2 qF | CTTGCACCAAGCAGCATGAA (SEQ ID NO: 17) |
| ACT2 qR | CCGATCCAGACACTGTACTTCCTT (SEQ ID NO: 18) |
| COR15a II F | ACGAAACGATTCTATTACAAGTAATGA (SEQ ID NO: 19) |
| COR15a II R | GCCCAAATGAGTTGAAACCACAAAC (SEQ ID NO: 20) |
| COR15a III F | GCCGACATACATTTGTTTCTATTACA (SEQ ID NO: 21) |
| COR 15a III R | GTGGTTTTCAGAAAGAAGAAGAAAAA (SEQ ID NO: 22) |
| TA3 FW | CTGCGTGGAAGTCTGTCAAA (SEQ ID NO: 23) |
| TA3 RW | CTATGCCACAGGGCAGTTTT (SEQ ID NO: 24) |

Primers for constructs used in BiFC assays

| | |
|---|---|
| Trx-h2 attB1 F | AAAAAGCAGGCCATATGGGAGGAGCTTTATCAAC (SEQ ID NO: 25) |
| Trx-h2 attB2 R | AGAAAGCTGGGTTATGCTCTGAGTTTGCTAA (SEQ ID NO: 26) |
| attB1 | GGGGACAAGTTTGTACAAAAAAGCAGGCCAT (SEQ ID NO: 27) |
| attB2 | GGGGACCACTTTGTACAAGAAAGCTGGGT (SEQ ID NO: 28) |
| CBF1 F | ATGAACTCATTTTCAGCTTTTTCTGAA (SEQ ID NO: 29) |
| CBF1 R | TTAGTAACTCCAAAGCGACACGTC (SEQ ID NO: 30) |
| CBF1 attB1 F | AAAAAGCAGGCCATATGAACTCATTTTCAG (SEQ ID NO: 31) |
| CBF1 attB1 R | AGAAAGCTGGGTTAGTAACTCCAAAGCGAC (SEQ ID NO: 32) |
| CBF2 F | ATGAACTCATTTTCTGCCTTTTCTGA (SEQ ID NO: 33) |
| CBF2 R | TTAATAGCTCCATAAGGACACGTCATC (SEQ ID NO: 34) |
| CBF2 attB1 F | AAAAAGCAGGCCATATGAACTCATTTTCTG (SEQ ID NO: 35) |
| CBF2 attB1 R | AGAAAGCTGGGTTAATAGCTCCATAAGGAC (SEQ ID NO: 36) |
| CBF3 F | ATGAACTCATTTTCTGCTTTTTCTGAAA (SEQ ID NO: 37) |
| CBF3 R | TTAATAACTCCATAACGATACGTCGTCAT(SEQ ID NO: 38) |
| CBF3 attB1 F | AAAAAGCAGGCCATATGAACTCATTTTCTG(SEQ ID NO: 35) |
| CBF3 attB1 R | AGAAAGCTGGGTTAATAACTCCATAACGAT(SEQ ID NO: 39) |

Primers for constructs for in vitro pull-down assay

| | |
|---|---|
| Trx-h2 F (BamHI) | GGATCCAATGGGAGGAGCTTTATCA (SEQ ID NO: 40) |
| Trx-h2 R (HindIII) | AAGCTTTTATGCTCTGAGTTTGCTAACTTT (SEQ ID NO: 41) |
| CBF1 F (EcoRI) | GAATTCATGAACTCATTTTCAGCT (SEQ ID NO: 42) |
| CBF1 R (HindIII) | AAGCTTTTAGTAACTCCAAAGCGA (SEQ ID NO: 43) |
| CBF2 F (EcoRI) | GAATTCATGAACTCATTTTCTGCCT (SEQ ID NO: 44) |
| CBF2 R (HindIII) | AAGCTTTTAATAGCTCCATAAGGACAC (SEQ ID NO: 45) |
| CBF3 F (EcoRI) | GAATTCATGAACTCATTTTCTGCTT (SEQ ID NO: 46) |
| CBF3 R (HindIII) | AAGCTTTTAATAACTCCATAACGAT (SEQ ID NO: 47) |

Primers for constructs for point-mutated CBF1

| | |
|---|---|
| CBF1 C23, 30S F | AGTCCGACGTTGGCCACGAGTAGT (SEQ ID NO: 48) |
| CBF1 C23, 30S R | ACTACTCGTGGCCAACGTCGGACT (SEQ ID NO: 49) |
| CBF1 C100S F | CAAGTCTCAACTTCGCTGACT (SEQ ID NO: 50) |
| CBF1 C100S R | AGTCAGCGAAGTTGAGACTTG (SEQ ID NO: 51) |
| CBF1 C117S F | AGCGCCAAGGATATCCAAA (SEQ ID NO: 52) |
| CBF1 C117S R | TTTGGATATCCTTGGCGCT (SEQ ID NO: 53) |
| CBF1 C137S F | CGAGTGATACGACGACCA (SEQ ID NO: 54) |
| CBF1 C137S R | TGGTCGTCGTATCACTCG (SEQ ID NO: 55) |

Primers for constructs for EMSA assay

| | |
|---|---|
| CRT/DRE F′ | ATTTCATGGCCGACCTGCTTTTT (SEQ ID NO: 56) |
| CRT/DRE R′ | AAAAAGCAGGTCGGCCATGAAAT (SEQ ID NO: 57) |

Primers for constructs for Transient transactivation assay

| | |
|---|---|
| P_{COR15a} F | TATTTCATATTGAATTAGGAGATGTTACTG (SEQ ID NO: 58) |
| P_{COR15a} R | GAAGCTGCTCTGCTTGGCTC (SEQ ID NO: 59) |
| P_{COR15a} attB1 F | AAAAAGCAGGCCATTATTTCATATTGAAT (SEQ ID NO: 60) |
| P_{COR15a} attB2 R | AGAAAGCTGAAGCTGCTCTGCTT (SEQ ID NO: 61) |

### REFERENCE TO AN ELECTRONIC SEQUENCE LISTING

A sequence listing electronically submitted on May 24, 2023 as a XML file named 20230524_S10923GR_05_TU_SEQ.TXT, created on May 2, 2023 and having a size of 27,311 bytes, is incorporated herein by reference in its entirety.

1. A transgenic plant the amino acid sequence of SEQ ID NO: 1, wherein the transgenic plant has cold resistance and overexpresses thioredoxin Trx-h2 protein that interacts with C-repeat-binding transcription factor 1 (CBF 1) having the amino acid sequence of SEQ ID NO: 2 to switch the CBF 1 from high molecular weight oligomers to low molecular weight monomers at temperatures of 0 to 10° C., thus conferring plant cold resistance.

2. The transgenic plant according to claim 1, wherein the thioredoxin Trx-h2 protein is myristoylated in the cytoplasm by glycine, the second amino acid residue, but it is demyristoylated from the cytoplasm at low temperatures of 0 to 10° C., and then it is translocated to a nucleus, interacting with CBF1 to cause structural switching of CBF1, thus conferring plant cold resistance.

3. The transgenic plant according to claim 2, wherein the thioredoxin Trx-h2 protein has a well-conserved Trx motif with 122 amino acids from the 31st residue to the 133rd residue, and cysteine, the 59th and 62nd residues in the Trx motif, interacts with CBF 1.

4. The transgenic plant according to claim 1, wherein the Cysteine, the 23, 30, 100, 117, and 137^{th} conserved residues in the CBF1, interacts with the thioredoxin Trx-h2 protein.

5. The transgenic plant according to claim 2, wherein the Cysteine, the 23, 30, 100, 117, and 137th conserved residues in the CBF1, interacts with the thioredoxin Trx-h2 protein.

6. The transgenic plant according to claim 3, wherein the Cysteine, the 23, 30, 100, 117, and 137th conserved residues in the CBF1, interacts with the thioredoxin Trx-h2 protein.
